SEQUENCE LISTING (6 sequences)

SEQ ID NO: 1 - protein, 640 aa, Homo sapiens
MGQPSLTWMLMVVVASWFITTAATDTSEARWCSECHSNATCTEDEAVTTCTCQEGFTGDGLTCVDLDECAIPGAHNCSANSSCVNTPGSFSCVCPEGFRLSPGLGCTDVDECAEPGLSHCHALATCVNVVGSYLCVCPAGYRGDGWHCECSPGSCGPGLDCVPEGDALVCADPCQAHRTLDEYWRSTEYGEGYACDTDLRGWYRFVGQGGARMAETCVPVLRCNTAAPMWLNGTHPSSDEGIVSRKACAHWSGHCCLWDASVQVKACAGGYYVYNLTAPPECHLAYCTDPSSVEGTCEECSIDEDCKSNNGRWHCQCKQDFNITDISLLEHRLECGANDMKVSLGKCQLKSLGFDKVFMYLSDSRCSGFNDRDNRDWVSVVTPARDGPCGTVLTRNETHATYSNTLYLADEIIIRDLNIKINFACSYPLDMKVSLKTALQPMVSALNIRVGGTGMFTVRMALFQTPSYTQPYQGSSVTLSTEAFLYVGTMLDGGDLSRFALLMTNCYATPSSNATDPLKYFIIQDRCPHTRDSTIQVVENGESSQGRFSVQMFRFAGNYDLVYLHCEVYLCDTMNEKCKPTCSGTRFRSGSVIDQSRVLNLGPITRKGVQATVSRAFSSLGLLKVWLPLLLSATLTLTFQ

SEQ ID NO: 2 - protein, 967 aa, Homo sapiens
MAKGFYISKSLGILGILLGVAAVCTIIALSVVYSQEKNKNANSSPVASTTPSASATTNPASATTLDQSKAWNRYRLPNTLKPDSYQVTLRPYLTPNDRGLYVFKGSSTVRFTCKEATDVIIIHSKKLNYTLSQGHRVVLRGVGGSQPPDIDKTELVEPTEYLVVHLKGSLVKDSQYEMDSEFEGELADDLAGFYRSEYMEGNVRKVVATTQMQAADARKSFPCFDEPAMKAEFNITLIHPKDLTALSNMLPKGPSTPLPEDPNWNVTEFHTTPKMSTYLLAFIVSEFDYVEKQASNGVLIRIWARPSAIAAGHGDYALNVTGPILNFFAGHYDTPYPLPKSDQIGLPDFNAGAMENWGLVTYRENSLLFDPLSSSSSNKERVVTVIAHELAHQWFGNLVTIEWWNDLWLNEGFASYVEYLGADYAEPTWNLKDLMVLNDVYRVMAVDALASSHPLSTPASEINTPAQISELFDAISYSKGASVLRMLSSFLSEDVFKQGLASYLHTFAYQNTIYLNLWDHLQEAVNNRSIQLPTTVRDIMNRWTLQMGFPVITVDTSTGTLSQEHFLLDPDSNVTRPSEFNYVWIVPITSIRDGRQQQDYWLIDVRAQNDLFSTSGNEWVLLNLNVTGYYRVNYDEENWRKIQTQLQRDHSAIPVINRAQIINDAFNLASAHKVPVTLALNNTLFLIEERQYMPWEAALSSLSYFKLMFDRSEVYGPMKNYLKKQVTPLFIHFRNNTNNWREIPENLMDQYSEVNAISTACSNGVPECEEMVSGLFKQWMENPNNNPIHPNLRSTVYCNAIAQGGEEEWDFAWEQFRNATLVNEADKLRAALACSKELWILNRYLSYTLNPDLIRKQDATSTIISITNNVIGQGLVWDFVQSNWKKLFNDYGGGSFSFSNLIQAVTRRFSTEYELQQLEQFKKDNEETGFGSGTRALEQALEKTKANIKWVKENKEVVLQWFTENSK

SEQ ID NO: 3 - protein, 766 aa, Homo sapiens
MKTPWKVLLGLLGAAALVTIITVPVVLLNKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 4 - protein, 750 aa, Homo sapiens
MGKSESQMDITDINTPKPKKKQRWTPLEISLSVLVLLLTIIAVTMIALYATYDDGICKSSDCIKSAARLIQNMDATTEPCTDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMTLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 5 - protein, 298 aa, Homo sapiens
MVRMVPVLLSLLLLLGPAVPQENQDGRYSLTYIYTGLSKHVEDVPAFQALGSLNDLQFFRYNSKDRKSQPMGLWRQVEGMEDWKQDSQLQKAREDIFMETLKDIVEYYNDSNGSHVLQGRFGCEIENNRSSGAFWKYYYDGKDYIEFNKEIPAWVPFDPAAQITKQKWEAEPVYVQRAKAYLEEECPATLRKYLKYSKNILDRQDPPSVVVTSHQAPGEKKKLKCLAYDFYPGKIDVHWTRAGEVQEPELRGDVLHNGNGTYQSWVVVAVPPQDTAPYSCHVQHSSLAQPLVVPWEAS

SEQ ID NO: 6 - protein, 341 aa, Homo sapiens
GDWYSDNEMPPEALSQGCKDIAYQLMHNIRDIDVIMGGGRKYMYPKNKTDVEYESDEKARGTRLDGLDLVDTWKSFKPRHKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSAGSLAAGPLLLALALYPLSVLF

(19) United States
(12) Patent Application Publication
(10) Pub. No.: US 2009/0220994 A1
(43) Pub. Date: Sep. 3, 2009

(54) METHODS FOR DIAGNOSIS OF CHRONIC PROSTATITIS/CHRONIC PELVIC PAIN SYNDROME

(75) Inventor: Jordan Dimitrakov, Boston, MA (US)

Correspondence Address: DAVID S. RESNICK, NIXON PEABODY LLP, 100 SUMMER STREET, BOSTON, MA, 02110-2131, US

(73) Assignee: CHILDREN'S MEDICAL CENTER CORPORATION, Boston, MA (US)

(21) Appl. No.: 12/393,357

(22) Filed: Feb. 26, 2009

Related U.S. Application Data
(60) Provisional application No. 61/032,119, filed on Feb. 28, 2008.

(51) Int. Cl. G01N 33/53

(52) U.S. Cl. 435/7.9; 435/7.1

(57) ABSTRACT

The present invention relates to methods for diagnosis of chronic prostatitis/chronic pain pelvic syndrome (CP/CPPS). We have found specific biomarkers that are present in higher concentrations in patients that have chronic prostatitis/chronic pain pelvic syndrome (CP/CPPS) as compared to subjects that have no symptoms of CP/CPPS. In particular, uromodulin (THP), aminopeptidase N (AMPN), dipeptidylpeptidase IV (CD26), neprilysin (NEP), zinc-α-2-glycoprotein (ZA2G) and alkaline phosphatase (ALP) were found to be present at higher concentrations in CP/CPPS patient urine that is voided after prostatic message. Accordingly, the invention is directed to methods for diagnosis of CP/CPPS by monitoring the levels of at least one of these proteins in post-prostatic massage urine, as well as to diagnostic kits designed for diagnosis of CP/CPPS.

### CROSS REFERENCE TO RELATED APPLICATION

This application claims benefit under 35 U.S.C. §119(e) of the U.S. provisional applications No. 61/032,119 filed on Feb. 28, 2008, the contents of which are incorporated herein by reference in their entirety.

### GOVERNMENT SUPPORT

This invention was made with Government support under grant number DK05990 awarded by the National Institute of Health. The Government has certain rights in the invention.

### FIELD OF THE INVENTION

The present invention relates generally to methods for the diagnosis of chronic prostatitis/chronic pain syndrome (CP/CPPS) by assessing the levels of novel biomarkers in prostatic fluid or post-prostatic massage urine.

### BACKGROUND OF THE INVENTION

Chronic prostatitis or chronic pelvic pain syndrome (CP/CPPS) is a debilitating condition characterized by pain or discomfort in the pelvic or perineal area and is a condition often associated with erectile dysfunction.

While CP/CPPS is a highly prevalent disease in men, with an estimated 35-50% of men affected by prostatitis at some time in life, definitive methods for the diagnosis and treatment of CP/CPPS have remained elusive.

Interstitial cystitis (IC) is the condition related to CP/CPPS in women. The chronic pain experienced by IC and CP/CPPS patients is similar to the pain experienced following injury, or inflammation of peripheral nerves. The stimuli that normally would never cause pain results in pain in these patients.

There is a need in the art to establish diagnostic and therapeutic markers to facilitate the diagnosis of this debilitating syndrome as well as to facilitate the development of therapeutics.

### SUMMARY OF THE INVENTION

The present invention is based on the discovery, via proteomics, of specific biomarkers that are present in higher concentrations in patients that have chronic prostatitis/chronic pain pelvic syndrome (CP/CPPS) as compared to subjects that have no symptoms of CP/CPPS. In particular, uromodulin (THP), aminopeptidase N (AMPN), dipeptidylpeptidase IV (CD26), neprilysin (NEP), zinc-α-2-glycoprotein (ZA2G) and alkaline phosphatase (ALP) were found to be present at higher concentrations in CP/CPPS patient urine that is voided after prostatic message. Accordingly, the invention is directed to methods for diagnosis of CP/CPPS by monitoring the levels of at least one of these proteins in post-prostatic massage urine, as well as to diagnostic kits designed for diagnosis of CP/CPPS.

In one embodiment, a method for facilitating the diagnosis of CP/CPPS is provided that comprises obtaining a post-prostatic massage urine sample from the subject and detecting the presence or absence of at least one biomarker protein in the post-prostatic massage urine sample, wherein the biomarker protein is selected form the group consisting of uromodulin (THP), aminopeptidase N (AMPN), dipeptidylpeptidase IV (CD26), neprilysin (NEP), zinc-α-2-glycoprotein (ZA2G) and alkaline phosphatase (ALP); and wherein the presence of at least one biomarker protein is indicative of the presence of CP/CPPS.

In one embodiment, the post-prostatic massage sample is compared to a control sample, wherein the control sample is a post-prostatic message urine sample from a subject that is asymptomatic for CP/CPPS.

In one embodiment, the presence at least two biomarkers is indicative of the presence of CP/CPPS.

In one embodiment, the presence at least three biomarkers is indicative of the presence of CP/CPPS.

In one embodiment, the presence at least four biomarkers is indicative of the presence of CP/CPPS.

In one embodiment, the presence at least five biomarkers is indicative of the presence of CP/CPPS.

In one embodiment, the presence at least six biomarkers is indicative of the presence of CP/CPPS.

In another embodiment, a method for facilitating the diagnosis of a subject for chronic prostatitis/chronic pain pelvic syndrome (CP/CPPS) is provided where protein levels of uromodulin (THP), aminopeptidase N (AMPN), dipeptidylpeptidase IV (CD26), neprilysin (NEP), zinc-α-2-glycoprotein (ZA2G) and alkaline phosphatase (ALP). The method comprises measuring the level of at least one biomarker protein in a post-prostatic massage urine test sample obtained from a patient and comparing the level of the biomarker measured in the test sample with the level of the same biomarker protein present in a control sample. A higher level of the measured biomarker protein in the test sample as compared to the level of the same biomarker protein in the control sample is indicative of CP/CPPS.

The term “test sample” refers to a urine sample obtained from a subject being tested for CP/CPPS after the subject has been subjected to prostatic massage. The prostatic massage results in prostatic fluid being voided in the test urine sample.

The term “control sample” refers to a pre-prostatic message urine sample obtained from the same subject. The term “control sample” also refers to post-prostatic message urine sample from a different subject that is asymptomatic for CP/CPPS.

In one embodiment, the levels of at least 2, 3, 4, 5, or 6 biomarker proteins are measured and higher levels of at least 2, 3, 4, 5, or 6 biomarker proteins in the test sample as compared to the levels of biomarker proteins in the control sample is indicative of CP/CPPS

In one aspect of the invention, levels of biomarker protein present in a test biological sample are measured by contacting the test sample, or preparation thereof, with an antibody-based binding moiety that specifically binds to the biomarker protein, or to a portion thereof. The antibody-based binding moiety forms a complex with the biomarker protein that can be detected, thereby allowing the levels of the biomarker protein to be measured.

Antibody-based immunoassays are the preferred means for measuring levels of biomarker protein, e.g. by ELISA assay. However, any means known to those skilled in art can be used to assess biomarker protein levels. Biomarker protein levels can be assessed by mass spectrometry, including SELDI mass spectrometry. Biomarker protein levels can also be assessed by a biological activity assay including, but not limited to, peptidase activity assays, e.g. to monitor levels of aminopeptidase N or dipeptidylpeptidase IV.

In a further embodiment, the invention provides for kits that comprise means for measuring the biomarker proteins in a urine sample to facilitate diagnosis of CP/CPPS.

The present invention further contemplates the assessment of levels of these biomarker proteins to monitor the therapeutic efficacy of a treatment regime designed to treat a patient having CP/CPPS.

Other aspects of the invention are disclosed infra.

### BRIEF DESCRIPTION OF THE DRAWINGS

The accompanying drawings, which are incorporated in and constitute a part of this specification, illustrate embodiments of the invention and, together with the description, serve to explain the objects, advantages, and principles of the invention.

FIG. 1 shows detection by Western blot of uromodulin (THP), aminopeptidase N (AMPN), dipeptidylpeptidase IV (CD26), neprilysin (NEP), and zinc-α-2-glycoprotein (ZA2G) in post-prostatic massage urine samples of patients that have CP/CPPS compared to post-prostatic massage urine control samples obtained from subjects that are asymptomatic for CP/CPPS.

FIG. 2 shows the amino acid sequence for uromodulin (THP) (SEQ ID NO:1).

FIG. 3 shows the amino acid sequence for aminopeptidase N (AMPN) (SEQ ID NO: 2).

FIG. 4 shows the amino acid sequence for dipeptidylpeptidase IV (CD26) (SEQ ID NO: 3).

FIG. 5 shows the amino acid sequence for neprilysin (NEP) (SEQ ID NO: 4).

FIG. 6 shows the amino acid sequence for zinc-α-2-glycoprotein (ZA2G) (SEQ ID NO: 5).

FIG. 7 shows the amino acid sequence for alkaline phosphatase (ALP) (SEQ ID NO: 6).

FIG. 8 shows a dot blot analysis of five of the markers—neprilysin (NEP), aminopeptidase N (AMPN, dipeptidylpeptidase IV (CD26), uromodulin (THP), and alkaline phosphatase (ALP). Comparisons were made between pre-massage (Pre) and post-prostatic massage (post) urine of each group and between CP/CPPS patients and controls.

FIG. 9 shows the EASE analysis using GO terms and KEGG pathways that revealed several pathways that were up-regulated in the post-prostatic M CP/CPPS protein set. The most significant GO terms found (P<0.001) and the most significant KEGG pathways (P<0.1) are shown.

FIG. 10A (top view) and 10B (side view) shows the schematic diagrams of a test strip for determining the level of a biomarker protein in a fluid sample and comparing the determined level with a reference value.

FIG. 11 shows a schematic diagram of the interpretation of the results obtained using the test strip shown in FIG. 10.

FIG. 12 shows a schematic diagram of how the levels of three biomarker proteins can be determined simultaneously using three test strips, one test strip for a different biomarker protein. A diagnostic kit can comprise several test strips, one strip for a different biomarker protein.

FIG. 13 shows a schematic diagram of how the levels of three biomarker proteins are determined simultaneously on the same membrane and test strip. A diagnostic kit can comprise a single composite test strip for determining the levels of several biomarker proteins simultaneously.

FIG. 14 shows a schematic diagram of an alternative version of a test strip for determining whether the level of a biomarker protein in a fluid sample is above or below a reference/control value for that biomarker.

FIG. 15A (top view) and 15B (side view) shows a schematic diagram of an alternative version of a test strip for determining the level of a biomarker protein in a fluid sample and comparing the determined level with a reference value. S, T, C definition are as in FIG. 10.

FIG. 16 shows a schematic diagram of the interpretation of the results obtained using the test strip shown in FIG. 15.

FIG. 17 shows a schematic diagram of an alternative version on how the levels of four biomarker proteins can be determined simultaneously using four separate test strips, one test strip for a different biomarker protein. A diagnostic kit can comprise multiple test strips, one strip for a different biomarker protein.

FIG. 18 shows a schematic diagram of an alternative version how the levels of three biomarker proteins are determined simultaneously on the same membrane and test strip. A diagnostic kit can comprise a single composite test strip for determining the levels of several biomarker proteins.

FIG. 19 shows a schematic diagram of an ELISA plate assay comprising standard protein curves.

FIG. 20 shows a schematic diagram of a modified ELISA plate assay utilizing fixed amounts of standard proteins.

### DETAILED DESCRIPTION OF THE INVENTION

Unless otherwise explained, all technical and scientific terms used herein have the same meaning as commonly understood by one of ordinary skill in the art to which this disclosure belongs. Definitions of common terms in urology, biochemistry and molecular biology can be found in The Merck Manual of Diagnosis and Therapy, 18th Edition, published by Merck Research Laboratories, 2006 (ISBN 0-911910-18-2); Robert S. Porter et al. (eds.); The Encyclopedia of Molecular Biology, published by Blackwell Science Ltd., 1994 (ISBN 0-632-02182-9), Robert A. Meyers (ed.); Molecular Biology and Biotechnology: a Comprehensive Desk Reference, published by VCH Publishers, Inc., 1995 (ISBN 1-56081-569-8); The ELISA guidebook (Methods in Molecular Biology 149) by Crowther J. R. (2000); Fundamentals of RIA and Other Ligand Assays by Jeffrey Travis, 1979, Scientific Newsletters; and Immunology by Werner Luttmann, published by Elsevier, 2006.

Unless otherwise stated, the present invention was performed using standard procedures, as described, for example in Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); A. R. Kimmerl Eds., Academic Press Inc., San Diego, USA (1987)) and Current Protocols in Immunology (CPI) (John E. Coligan, et. al., ed. John Wiley and Sons, Inc.), which are all incorporated by reference herein in their entireties.

Methods for the production of antibodies are disclosed in PCT publication WO 97/40072 or U.S. Application. No. 2002/0182702, which are herein incorporated by reference. The processes of immunization to elicit antibody production in a mammal, the generation of hybridomas to produce monoclonal antibodies, and the purification of antibodies may be performed by described in “Current Protocols in Immunology” (CPI) (John Wiley and Sons, Inc.) and Antibodies: A Laboratory Manual (Ed Harlow and David Lane editors, Cold Spring Harbor Laboratory Press 1988) which are both incorporated by reference herein in their entireties.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages may mean ±1%.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The inventors have discovered that the levels of specific protein biomarkers present in post-prostatic massage urine samples of human subjects correlate with the presence, or absence of, chronic prostatitis/chronic pelvic pain syndrome (CP/CPPS) in the subject. These protein biomarkers are uromodulin (THP), aminopeptidase N (AMPN), dipeptidylpeptidase IV (CD26), neprilysin (NEP), zinc-α-2-glycoprotein (ZA2G) and alkaline phosphatase (ALP).

Accordingly, the present invention is directed to methods for facilitating diagnosis of CP/CPPS in a patient. In one embodiment the method comprises obtaining a post-prostatic massage urine sample from the subject and detecting the presence or absence of at least one biomarker protein in the post-prostatic massage urine sample, wherein the biomarker protein is selected form the group consisting of uromodulin (THP), aminopeptidase N (AMPN), dipeptidylpeptidase IV (CD26), neprilysin (NEP), zinc-α-2-glycoprotein (ZA2G) and alkaline phosphatase (ALP); and wherein the presence of at least one biomarker protein is indicative of the presence of CP/CPPS

In another embodiment, the methods involve measuring levels of biomarker protein in a test sample obtained from a patient, suspected of having CP/CPPS, and comparing the observed levels to levels of biomarker protein found in a control sample, for example a sample obtained from an individual patient or population of individuals that are asymptomatic for CP/CPPS. Levels of biomarker protein higher than levels that are observed in the normal control indicate the presence of CP/CPPS. The levels of biomarker protein can be represented by arbitrary units, for example as units obtained from a densitometer, luminometer, an activity assay, or an ELISA plate reader.

For purposes of comparison, the control sample can also be a standard sample that contains the same concentration of biomarker protein that is normally found in a post-prostatic massage urine sample that is obtained from a healthy individual. For example, there can be a standard normal control sample for the amounts of biomarker protein.

In one aspect, the control sample is a standard sample representative of the average level of each of the biomarker proteins in a post-prostatic massage urine sample from a pollutions of healthy human males not having any symptoms known to be associated with CP/CPPS.

In one aspect of the invention, a secondary diagnostic step can be performed. For example, if a level of biomarker protein is found to indicate the presence of CP/CPPS, then an additional method of detecting the syndrome can be performed to confirm the presence of CP/CPPS. Any of a variety of additional diagnostic steps can be used, such as direct analysis of prostatic fluid, or any other method.

In another embodiment, the invention provides a kit designed for facilitating the diagnosis of chronic prostatitis/chronic pelvic pain syndrome (CP/CPPS) in a human comprising: (a) at least one solid support; (b) at least three antibody-based moieties specific for each of three different biomarker proteins, wherein the three biomarker proteins comprises uromodulin (THP), aminopeptidase N (AMPN), and neprilysin (NEP); (c) at least three labeled antibody-based moieties specific for each of the three different biomarker proteins; and (d) instruction for use and interpretation of the kit. Such a kit allows the determination of the levels of THP, AMPN and NEP from a post-prostatic massage urine sample using an antibody-based method (e. g. ELISA).

In one embodiment, the kit further comprises (a) a fourth antibody-based moiety specific for a fourth biomarker protein, wherein the fourth biomarker protein is selected from the group consisting of dipeptidylpeptidase (CD26), zinc-α-2-glycoprotein (ZA2G) and alkaline phosphatase (ALP); and (b) a fourth labeled antibody-based moiety specifically binding the fourth biomarker protein. For example, such a kit would allow the determination of the levels of THP, AMPN, NEP and CD26.

In another embodiment, the kit further comprises (a) a fifth antibody-based moiety specific for a fifth biomarker protein, wherein the fifth biomarker protein is selected from the group consisting of dipeptidylpeptidase (CD26), zinc-α-2-glycoprotein (ZA2G) and alkaline phosphatase (ALP), wherein the fourth and fifth biomarker proteins selected are different and (b) a fifth labeled antibody-based moiety specifically binding the fifth biomarker protein. For example, such a kit would allow the determination of the levels of THP, AMPN, NEP, CD26 and ZA2G.

In another embodiment, the kit comprises (a) a sixth antibody-based moiety specific for a sixth biomarker protein, wherein the sixth biomarker protein is selected from the group consisting of dipeptidylpeptidase (CD26), zinc-α-2-glycoprotein (ZA2G) and alkaline phosphatase (ALP); wherein the fourth, fifth and sixth biomarker proteins are different; and (b) a sixth labeled antibody-based moiety specific for said sixth biomarker protein. Such a kit would allow the determination of the levels of all six biomarkers: THP, AMPN, NEP, CD26, ALP and ZA2G.

In another embodiment, the kit comprises (a) at least one solid support; (b) at least one antibody-based moiety specific for a biomarker protein, wherein the biomarker protein is selected from the group consisting of uromodulin (THP), aminopeptidase N (AMPN), neprilysin (NEP), dipeptidylpeptidase (CD26), zinc-α-2-glycoprotein (ZA2G) and alkaline phosphatase; (c) at least one labeled antibody-based moiety specific for the biomarker protein of (b); and (d) instruction for use and interpretation of the kit.

In another embodiment, the kit comprises (a) at least one solid support; (b) at least two antibody-based moieties specific for two different biomarker proteins, wherein the biomarker proteins are selected from the group consisting of uromodulin (THP), aminopeptidase N (AMPN), neprilysin (NEP), dipeptidylpeptidase (CD26), zinc-α-2-glycoprotein (ZA2G) and alkaline phosphatase; (c) at least two labeled antibody-based moieties specific for the biomarker proteins of (b); and (d) instruction for use and interpretation of the kit.

In another embodiment, the kit comprises (a) at least one solid support; (b) at least three antibody-based moieties specific for three different biomarker proteins, wherein the biomarker proteins are selected from the group consisting of uromodulin (THP), aminopeptidase N (AMPN), neprilysin (NEP), dipeptidylpeptidase (CD26), zinc-α-2-glycoprotein (ZA2G) and alkaline phosphatase; (c) at least three labeled antibody-based moieties specific for the biomarker proteins of (b); and (d) instruction for use and interpretation of the kit.

In another embodiment, the kit comprises (a) at least one solid support; (b) at least four antibody-based moieties specific for four different biomarker proteins, wherein the biomarker proteins are selected from the group consisting of uromodulin (THP), aminopeptidase N (AMPN), neprilysin (NEP), dipeptidylpeptidase (CD26), zinc-α-2-glycoprotein (ZA2G) and alkaline phosphatase; (c) at least four labeled antibody-based moieties specific for the biomarker proteins of (b); and (d) instruction for use and interpretation of the kit.

In another embodiment, the kit comprises (a) at least one solid support; (b) at least five antibody-based moieties specific for five biomarker proteins, wherein the biomarker proteins are selected from the group consisting of uromodulin (THP), aminopeptidase N (AMPN), neprilysin (NEP), dipeptidylpeptidase (CD26), zinc-α-2-glycoprotein (ZA2G) and alkaline phosphatase; (c) at least five labeled antibody-based moieties specific for the biomarker proteins of (b); and (d) instruction for use and interpretation of the kit.

In another embodiment, the kit comprises (a) at least one solid support; (b) at least six antibody-based moieties specific for each of biomarker proteins, uromodulin (THP), aminopeptidase N (AMPN), neprilysin (NEP), dipeptidylpeptidase (CD26), zinc-α-2-glycoprotein (ZA2G) and alkaline phosphatase; (c) at least six labeled antibody-based moieties specific for the biomarker proteins of (b); and (d) instruction for use and interpretation of the kit.

In some embodiments, for the kits described herein, the solid support is contacted with a sample from a subject. The sample is a post-prostatic massage urine sample and the subject is a human male.

In some embodiments, the kits described herein further comprise standards of known amounts of the biomarker proteins. In other embodiments, the kits described herein further comprise reference values of the levels of biomarker proteins. These reference values allow the determination of whether the levels of biomarker proteins in a post-prostatic massage urine sample is at least about two fold greater from the reference levels; wherein at least about two fold greater than the reference level indicates that the likelihood of CP/CPPS in the human. Reference values can be provided as numerical values, or as standards of known amounts of biomarkers.

In some embodiments, the reference values are average levels of each of the biomarker protein in post-prostatic massage urine samples from a population of healthy male humans not having CP/CPPS. Not having CP/CPPS means that the male is not suffering from CP/CPPS, has not been diagnosed with CP/CPPS and/or have no overt symptoms known to be associated with the current clinical diagnosis of CP/CPPS. A population of healthy male humans comprises at least 5 individuals, preferably 20 or more individuals. The average level can be obtained by dividing the total levels of biomarker from the population by the number of individuals in that population.

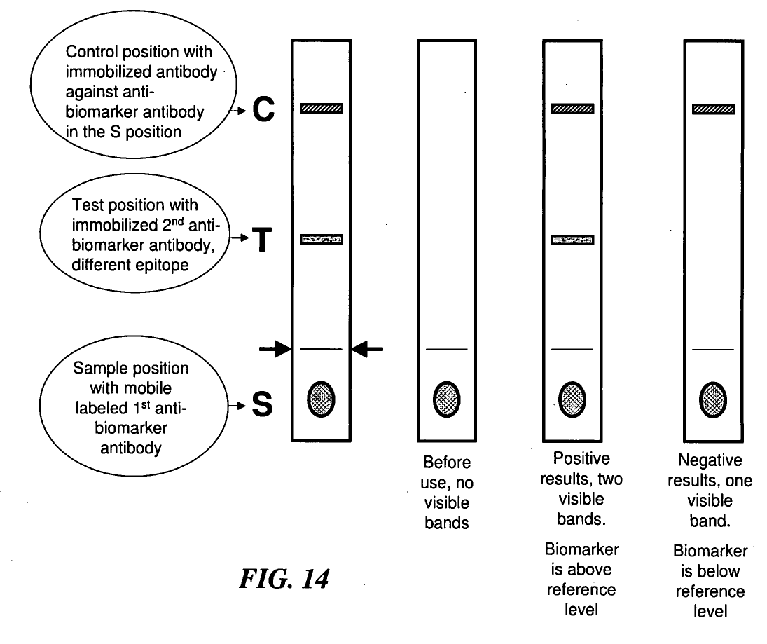

In one embodiment, for the kits described herein, at least one of the antibody-based moieties is immobilized on at least one solid support. (See Example 3 and, FIG. 14).

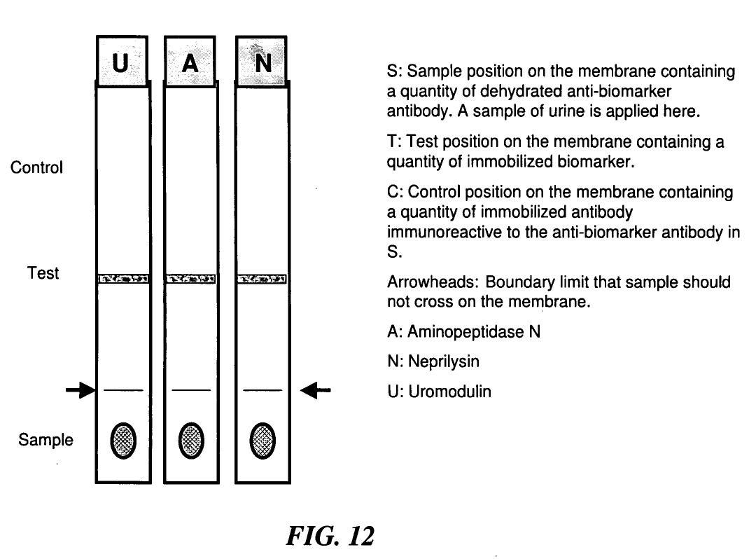

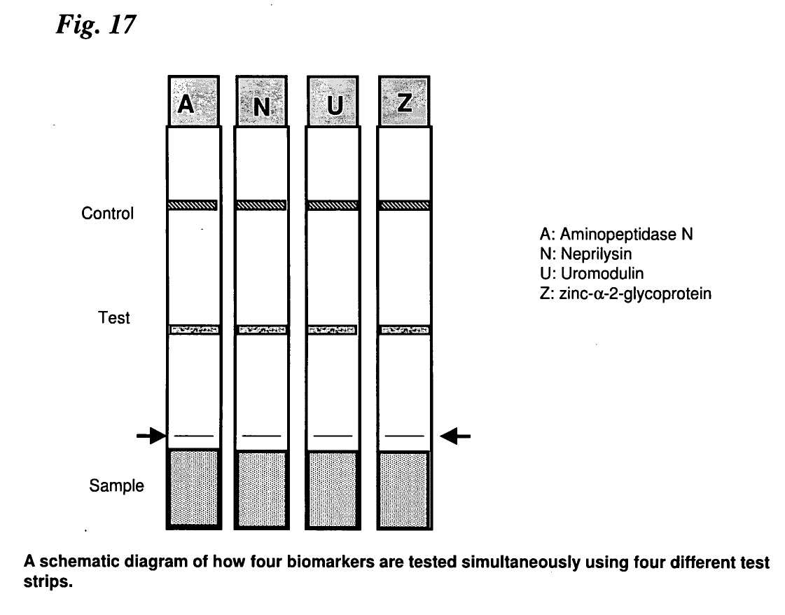

In some embodiments of the kits described herein, at least one of the antibody-based moieties is immobilized on a first solid support and at least another of the antibody-based moieties is immobilized on a second solid support (see Example 2 and 4, the test strips illustrated in FIGS. 12 and 17).

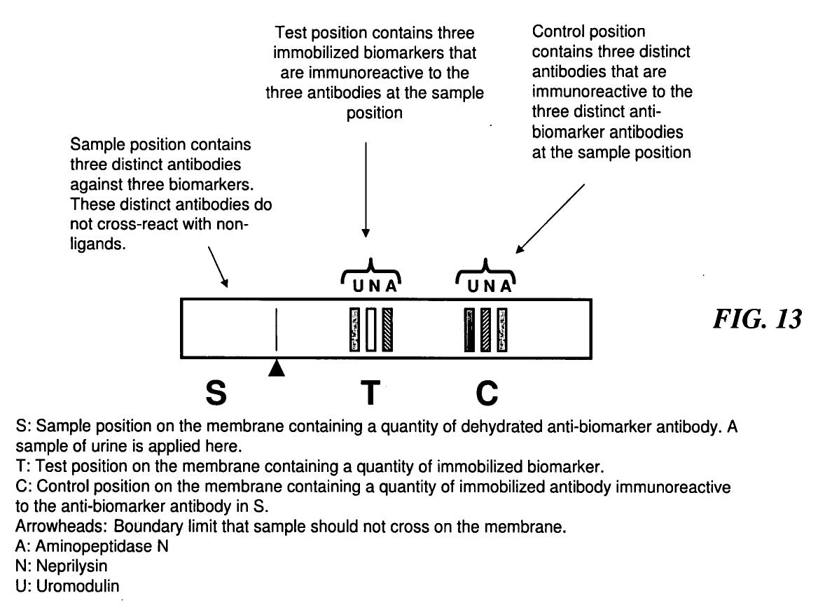

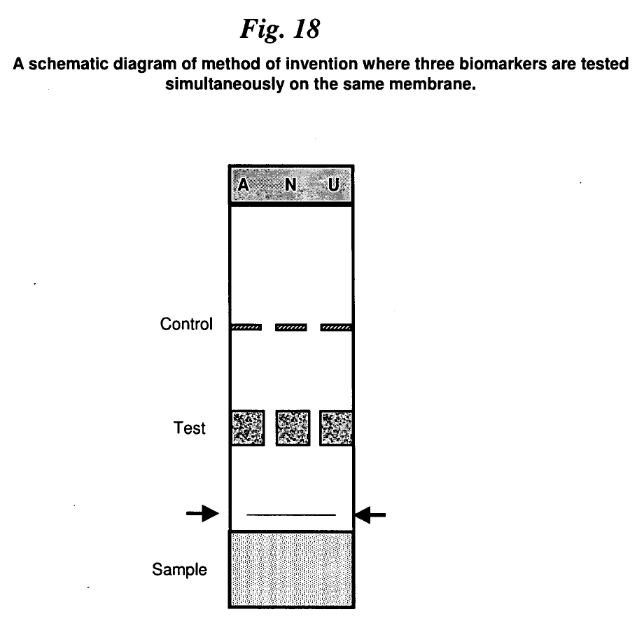

In some embodiments of the kits described herein, each of the antibody-based moiety is immobilized on the same solid support, such as shown in the test strips illustrated in FIG. 13 and FIG. 18. Such a kit allows simultaneous testing of more than one biomarker at one time.

In one embodiment of the kits described herein, at least one of the antibody-based moieties is disposed upon but not immobilized on at least one solid support. Such the antibody-based moiety is mobile on the solid support, e. g. when in an aqueous solution.

Any solid support can be used, including but not limited to, nitrocellulose membrane, nylon membrane, solid organic polymers, such as polystyrene, solid beads or laminated dipsticks such as described in U.S. Pat. No. 5,550,375. The use of “dip sticks” or test strips and other solid supports have been described in the art in the context of an immunoassay for a number of antigens. Three U.S. patents (U.S. Pat. No. 4,444,880, issued to H. Tom; U.S. Pat. No. 4,305,924, issued to R. N. Piasio; and U.S. Pat. No. 4,135,884, issued to J. T. Shen) describe the use of “dip stick” technology to detect soluble antigens via immunochemical assays. The apparatuses and methods of these three patents broadly describe a first component fixed to a solid surface on a “dip stick” which is exposed to a solution containing a soluble antigen that binds to the component fixed upon the “dip stick,” prior to detection of the component-antigen complex upon the stick.

Examples of kits includes but are not limited to ELISA assay kits, and kits comprising test strips and dipsticks. In an ELISA kit, an excess amount of antibody-based moieties specific for a particular antigen, in this case, a biomarker, is immobilized on a solid support. A sample containing an unknown amount of biomarker of interest is added to the immobilized antibody-based moiety, resulting in the formation of a complex consisting of the biomarker and the antibody-based moiety. The complex is detected by a labeled second antibody-based moiety that is also specific for the biomarker. The amount of label detected is a measure of the amount of biomarker present in the sample (see Example 5 and 6).

In some embodiments of the kits described herein, the kit comprises a test strip or a dipstick.

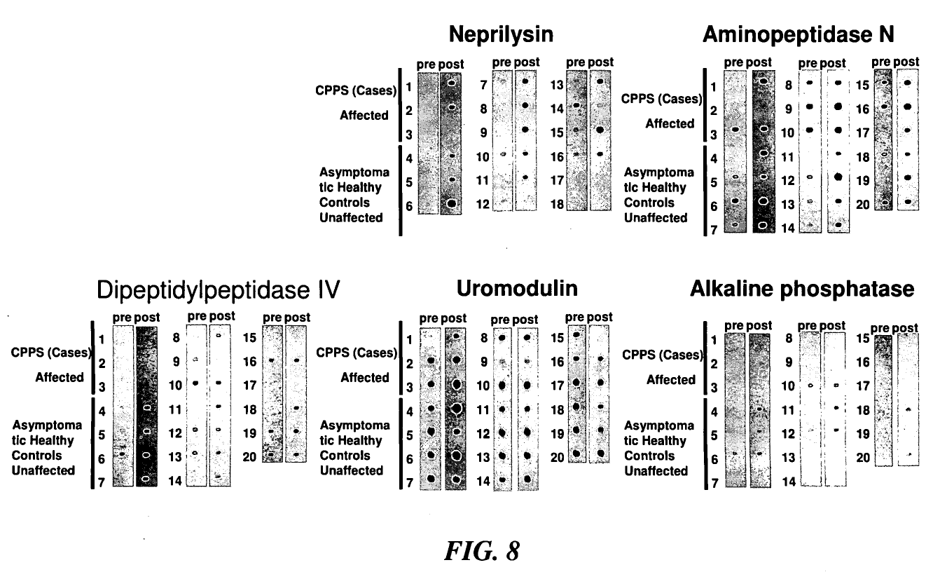

In some embodiments of the kits described herein, the kit comprises a dot blot as shown in Example 1, FIG. 8.

In some embodiments, when the solid support of the kits described herein is contacted with a sample containing a biomarker of interest, an antibody-based moiety: biomarker complex is formed. The sample is a post-prostatic massage urine. In some embodiments, the antibody-based moiety: biomarker complexes are detected.

In some embodiments of the kits described herein, the labeled antibody-based moieties are detectably labeled. In some embodiments, the detectable label is selected from a group consisting of enzyme, fluorescent, biotin, gold, latex, hapten and radioisotope labeling. A detectable hapten include but are not limited to biotin, fluorescein, digoxigenin, dinitrophenyl (DNP). Other labels include but are not limited to colloidal gold and latex beads. The latex beads can also be colored. Method of labeling antibodies, antibody-based moiety, or proteins are known in the art, for example, as described in “Colloidal Gold. Principles. Methods and Applications”, Hayat M A (ed) (1989-91). Vols 1-3, Academic press, London; in “Techniques in Immunocytochemistry”, Bullock G R and Petrusz P (eds) (1982-90) Vols 1, 2, 3, and 4, Academic Press, London; in “Principles of Biological Microtechnique”, Baker J R (1970), Methuen, London; Lillie R D (1965), Histopathologic Technique and practical Histochemistry, 3rd ed, McGraw Hill, New York; Berryman M A, et al (1992), J. Histochem Cytochem 40, 6, 845-857, all of which are incorporated hereby reference in their entirety.

In colloidal gold labeling technique, the unique red color of the accumulated gold label, when observed by lateral or transverse flow along a membrane on which an antigen is captured by an immobilized antibody, or by observation of the red color intensity in solution, provides an extremely sensitive method for detecting sub nanogram quantities of proteins in solution. A colloidal gold conjugate consists of a suspension of gold particles coated with a selected protein or macromolecule (such as an antibody or antibody-based moiety). The gold particles may be manufactured to any chosen size from 1-250 nm. This gold probe detection system, when incubated with a specific target, such as in a tissue section, will reveal the target through the visibility of the gold particles themselves. For detection by eye, gold particles will also reveal immobilized antigen on a solid phase such as a blotting membrane through the accumulated red color of the gold sol. Silver enhancement of this gold precipitate also gives further sensitivity of detection. Suppliers of colloidal gold reagents for labeling are available from SPI-MARK™. Polystyrene latex Bead size 200 nm colored latex bead coated with antibody SIGMA ALDRICH®, Molecular Probes, Bangs Laboratory Inc., and AGILENT® Technologies.

In other embodiments of the kits described herein, at least one of the labeled antibodies comprises an enzyme-labeled antibody-based moiety. The biomarker of interest that is bound and captured by the immobilized antibody-based moiety on the solid support (e. g. microtiter plate wells) is identified by adding a chromogenic substrate for the enzyme conjugated to the anti-antibody-based moiety and color production detected by an optical device such as an ELISA plate reader.

Other detection systems can also be used, for example, a biotin-streptavidin system. In this system, one of the antibodies (either the antibody-based moiety immunoreactive (i. e. specific for) with the biomarker of interest or the antibody-based moiety immunoreactive with that specific antibody) is biotinylated. The non-biotinylated antibody are incubated with wells coated with the biomarker antigen. Quantity of biotinylated antibody bound to the coated biomarker is determined using a streptavidin-peroxidase conjugate and a chromagenic substrate. Such streptavidin peroxidase detection kits are commercially available, e. g. from DAKO; Carpinteria, Calif.

Antibodies and antibody-based moiety can alternatively be labeled with any of a number of fluorescent compounds such as fluorescein isothiocyanate, europium, lucifer yellow, rhodamine B isothiocyanate (Wood, P. In: Principles and Practice of Immunoasay, Stockton Press, New York, pages 365-392 (1991)) for use in immunoassays. In conjunction with the known techniques for separation of antibody-antigen complexes, these fluorophores can be used to quantify the biomarker of interest. The same applies to chemiluminescent immunoassay in which case antibody or biomarker of interest can be labeled with isoluminol or acridinium esters (Krodel, E. et al., In: Bioluminescence and Chemiluminescence: Current Status. John Wiley and Sons Inc. New York, pp 107-110 (1991); Weeks, I. et al., Clin. Chem. 29:1480-1483 (1983)). Radioimmunoassay (Kashyap, M. L. et al., J. Clin. Invest, 60:171-180 (1977)) is another technique in which antibody can be used after labeling with a radioactive isotope such as 1251. Some of these immunoassays can be easily automated by the use of appropriate instruments such as the IMX™ (Abbott, Irving, Tex.) for a fluorescent immunoassay and Ciba Coming ACS 180™ (Ciba Corning, Medfield, Mass.) for a chemiluminescent immunoassay.

In some embodiments, the kits described herein further comprise at least one sample collection container for sample collection. Collection devices and container include but are not limited to sample containers urine samples.

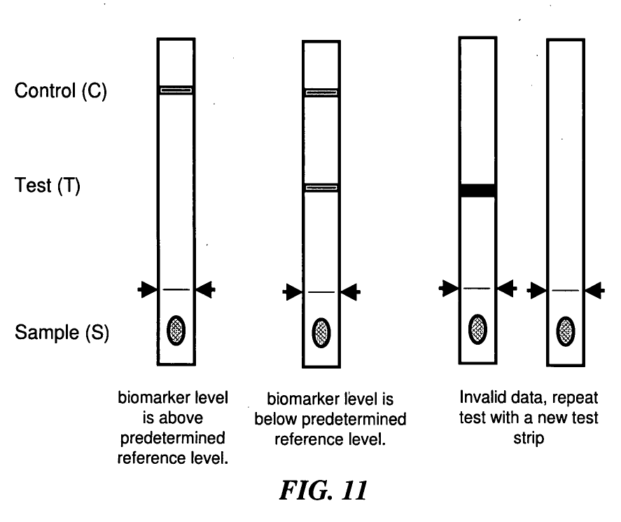

In some embodiments, the kits described herein further comprise instructions for using the kit and interpretation of results. For example, a chart showing FIG. 11, 14 or 16 for interpretation of results.

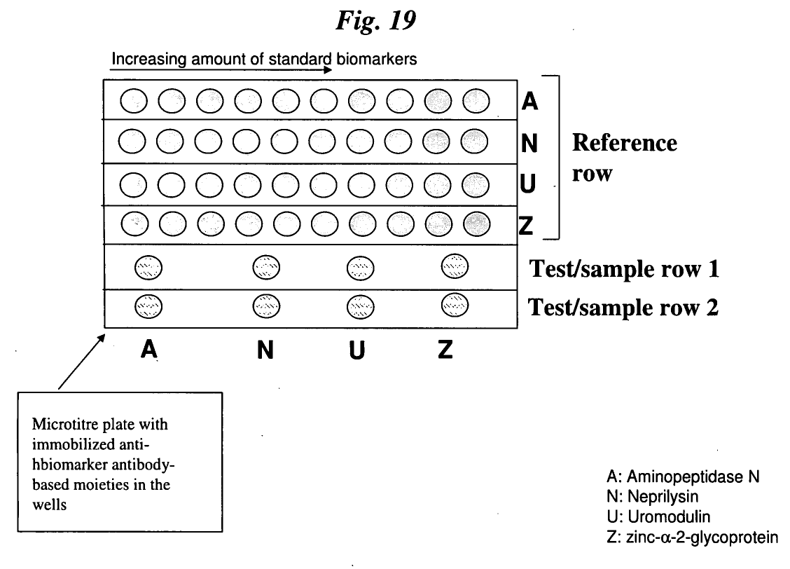

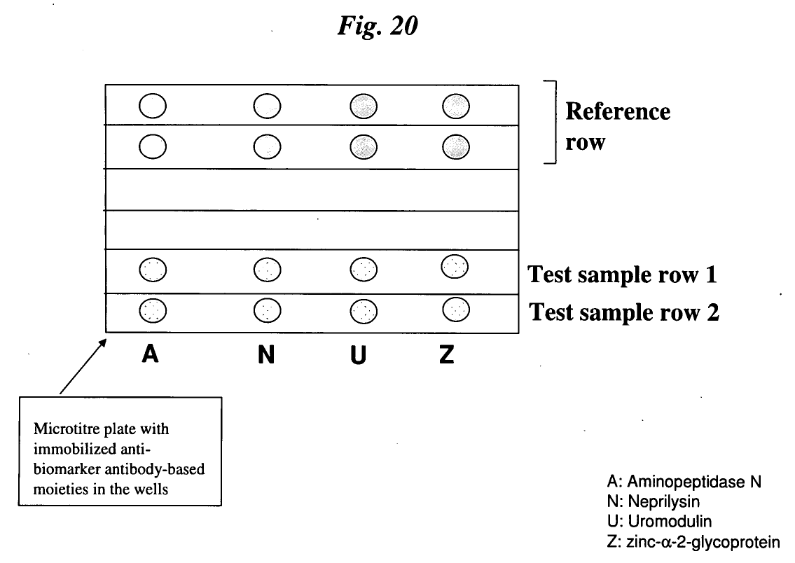

As an exemplary, using a typical ELISA-based assay kit would involved dispensing a sample containing the biomarker of interest into microtiter plate wells, preferably in duplicates or triplicates (as in FIGS. 19 and 20). The wells are coated with immobilized antibody-based moiety specific for the biomarker of interest. Alternatively, the ELISA plates pre-coated with immobilized antibody-based moiety specific for the biomarker of interest and/or control, non-biomarker reactive antibody can be provide with the ELISA kit. Next, a fixed amount of the standard biomarker provided with the kit is also dispensed into reference wells in the microtiter plate, also preferably in duplicates or triplicates, according the kit's instruction. That fixed amount of the standard biomarker corresponding to two fold of the reference value of the biomarker normally present in healthy men. Subsequently, the labeled antibody-based moiety that is also specific for that biomarker is added to both sample and reference wells. This is a “sandwich” ELISA assay, where the biomarker of interest is sandwich between two different antibody-based moieties that are specific for that biomarker. Since the amount of label detected is a measure of the amount of biomarker present in the wells, the amounts of label detected in the various wells provides means for comparing the level of the biomarker of interest in the sample with the reference value of the biomarker normally present in healthy men. For example, if the label is colored latex beads, a greater color intensity in the sample wells compared to the reference wells indicates that the level of the biomarker in the sample is higher than two fold of the reference value of the biomarker normally present in healthy men.

In one embodiment, the invention provides a method for facilitating the diagnosis of a subject for chronic prostatitis/chronic pain pelvic syndrome (CP/CPPS) comprising: (a) obtaining a post-prostatic massage urine sample from the subject; and (b) detecting the presence of at least one biomarker protein in the post-prostatic massage urine sample, wherein the biomarker protein is selected form the group consisting of uromodulin (THP), aminopeptidase N (AMPN), dipeptidylpeptidase IV (CD26), neprilysin (NEP), zinc-α-2-glycoprotein (ZA2G) and alkaline phosphatase (ALP); and wherein the presence of at least one biomarker protein is indicative of the presence of CP/CPPS.

In one embodiment, the post-prostatic massage sample is compared to a control sample, wherein the control sample is a post-prostatic message urine sample from a subject that is asymptomatic for CP/CPPS.

In one embodiment, the presence of at least two biomarkers is indicative of the presence of CP/CPPS. For example, CD26 and NEP.

In other embodiments, the presence of at least three biomarkers, at least four biomarkers, at least five biomarkers, and all six biomarkers is indicative of the presence of CP/CPPS.

In another embodiment, the invention provides a method for facilitating the diagnosis of a subject for chronic prostatitis/chronic pain pelvic syndrome (CP/CPPS) comprising: (a) measuring the level of at least one biomarker protein in a post-prostatic massage urine test sample obtained from the patient; and (b) comparing the level of the biomarker measured in the test sample with the level of the same biomarker protein present in a control/reference sample; wherein a higher level of the measured biomarker protein in the test sample as compared to the level of the same biomarker protein in the control/reference sample is indicative of CP/CPPS, and wherein the biomarker protein is selected form the group consisting of uromodulin (THP), aminopeptidase N (AMPN), dipeptidylpeptidase IV (CD26), neprilysin (NEP), zinc-α-2-glycoprotein (ZA2G) and alkaline phosphatase (ALP).

In one embodiment, the biomarker exceeds the control/reference sample value by at least 2 fold. In one embodiment, the biomarker exceeds the reference value by at least 3 fold. In one embodiment, the biomarker exceeds the reference value by at least 4 fold. In some embodiments, the biomarker exceeds the reference value by at least 8 fold, at least 10 fold, at least 12 fold, at least 15 fold, at least 20 fold, at least 25 fold, at least 30 fold or more. The number of fold that the biomarker level exceed that of the reference value can vary from 2 to 30 to 100, and can even be well beyond 100. Intermediate folds between 2 and 100 are included. In some other embodiments, the biomarker exceeds the reference value by hundreds of folds. In some embodiments, the greater the number folds over that of the reference value indicates the increased severity of CP/CPPS in the human.

In one embodiment, the presence of the biomarker protein is detected using an antibody-based moiety which specifically binds to the biomarker protein. In one embodiment, the binding results in the formation of an antibody-based moiety: biomarker complex. In one embodiment, the complex is detectably labeled for quantification.

In one embodiment, the levels of at least 2, 3, 4, 5, or 6 biomarker proteins are measured and higher levels of at least 2, 3, 4, 5, or 6 biomarker proteins in the test sample as compared to the levels of biomarker proteins in the control/reference sample is indicative of CP/CPPS.

In one embodiment, the level of the biomarker protein is measured by measuring the activity of the biomarker.

In one embodiment, the level of biomarker protein is determined an antibody-based analytical method, e. g. Western blot analysis or enzyme-linked immunosorbent assay (ELISA), wherein an antibody-based moiety:biomarker complex is formed and detected. The level of biomarker protein is measured by a method comprising the steps of: (a) contacting the test sample, or preparation thereof, with an antibody-based binding moiety which specifically binds the biomarker protein to form an antibody-biomarker protein complex; and (b) detecting the presence of the complex, thereby measuring the level of biomarker protein present.

In one embodiment, the level of biomarker protein is determined by mass spectrometry. In one embodiment, the quantification by mass spectrometry comprises ionizing a biomarker, separating the ionized biomarker according to its mass (m)-to-charge (z) ratios (m/z), and detecting the ionized biomarker. In addition, the protein biomarker are enzymatically digested into smaller peptides using a protease such as trypsin prior to ionization, separation, detection and analyses.

In one embodiment, the antibody-based binding moiety is labeled with a detectable label. The label is selected from the group consisting of a radioactive label, a hapten label, a fluorescent label, and an enzymatic label.

In one embodiment, the antibody-based binding moiety is an antibody.

In one embodiment, the antibody is an monoclonal antibody.

### Definitions

As used herein, “a higher level of biomarker protein in the test sample as compared to the level in the control/reference sample” refers to an amount of biomarker protein that is greater than an amount of biomarker protein present in a control/reference sample. The term “higher level” refers to a level that is statistically significant or significantly above levels found in the control sample. Preferably, the “higher level” is at least 2 fold greater.

As used herein, “biomarker protein” refers to a protein selected from the group consisting of uromodulin (THP), aminopeptidase N (AMPN), dipeptidylpeptidase IV (CD26), neprilysin (NEP), zinc-α-2-glycoprotein (ZA2G) and alkaline phosphatase (ALP).

The term “statistically significant” or “significantly” refers to statistical significance and generally means a two standard deviation (2SD) above normal, or higher, concentration of the marker.

As used herein, the term “comprising” or “comprises” is used in reference to methods and kits means that are essential to the invention, yet open to the inclusion of unspecified elements, whether essential or not. The use of “comprising” indicates inclusion rather than limitation.

As used herein, the term “consisting of” refers to methods and kits as described herein, which are exclusive of any element not recited in that description of the embodiment.

As used herein, the term “healthy men” refers to male humans between 18-60 years old who exhibit no symptoms that are known to associated with CP/CPPS, such as those described herein.

As used herein, the term “specific for a biomarker protein” or “specific to the protein” with respect to antibodies or antibody-based moieties means that antibodies or antibody-based moiety bind specifically and immunologically to the particular biomarker being referenced. In other words, the antibodies or antibody-based moieties are immunogenic or immunoreactive to the biomarker, while not binding significantly to other biomarkers or proteins in a particular assay.

### Biomarker Proteins of the Invention

As used herein, “Uromodulin (THP)” refers to the uromodulin protein of Genbank accession, protein, NP_{—}001008390, UniProt/Swiss-Prot: UROM_HUMAN, P07911 (Homo sapiens) (SEQ ID NO:1). The term “THP Uromodulin” also encompasses species variants, homologues, allelic forms, mutant forms, and equivalents thereof. In the methods described herein, fragments of these proteins can be detected. Uromodulin, also known as UMOD, Tamm-Horsfall protein (THP), or Tamm-Horsfall mucoprotein, is the most abundant protein in normal urine. Its excretion in urine follows proteolytic cleavage of the ectodomain of its glycosyl phosphatidylinosital-anchored counterpart that is situated on the luminal cell surface of the loop of Henle.

Methods of determining the conditions for solubilization of uromodulin in human urine and enzyme-linked immunosorbent assay (ELISA) for uromodulin are known in the art, e. g. as described by Kobayashi and Fukuoka S. in Arch Biochem Biophys 2001; 388: 113-120. Commercial antibodies against human uromodulin are available from R and D Systems, catalog Nos. AF5144 and BAF5144.

“Aminopeptidase N (AMPN)” refers to the Aminopeptidase N protein of Genbank accession, NP_{—}001141 protein, UniProtKB/Swiss-Prot P15144 (Homo sapiens) (SEQ ID NO:2). The term “aminopeptidase N” also encompasses species variants, homologues, allelic forms, mutant forms, and equivalents thereof. In the methods described herein, fragments of these proteins can be detected. Aminopeptidase N (APN; EC 3.4.11.2), also known as CD13, PEPN, gp150, p150, or APN is a Zn²⁺-dependent ectopeptidase that degrades preferentially proteins with an NH₂-terminal neutral amino acid. Although aminopeptidase N is a membrane protein, human plasma contains significant amounts of an active soluble form of aminopeptidase N (van Hensbergen et al, Clin. Cancer res. 8, 3747-3754). The ectopeptidase aminopeptidase N (APN, CD13, E.C.3.4.11.2) is expressed on endothelial cells, cells of the myelo-monocytic lineage, activated T cells, fibroblasts and keratinocytes. In addition to its proteolytic function it is involved in the regulation of various physiologic functions such as proliferation, cytokine production, cell-cell interaction and angiogenesis.

Commercial antibodies against AMPN are available from R and D Systems, catalog No. AF3815 and MAB3815. WO/2006/119887 describes a method of making antibody against AMPN and the use of the antibodies for quantifying AMPN by ELISA.

“Dipeptidylpeptidase IV (CD26)” refers to the dipeptidylpeptidase IV protein of Genbank accession, protein, NP AAA51943 (Homo sapiens) (SEQ ID NO:3). The term “Dipeptidylpeptidase IV (CD26)” also encompasses species variants, homologues, allelic forms, mutant forms, and equivalents thereof. In the methods described herein, fragments of these proteins can be detected. Dipeptidylpeptidase IV is also known as DPP4; dipeptidyl-peptidase 4; adenosine deaminase complexing protein 2 (ADABP); ADCP2; CD26; DPPIV; TP103; and T-cell activation antigen CD26.

Commercial antibodies against CD26 are available from RayBiotech Inc. and commercial ELISA kits are available from INVITROGEN Inc. catalog No. SKU# KHS6021 (CD26 (soluble) Human ELISA Kit).

“Neprilysin (NEP)” refers to the Neprilysin (NEP)” protein of Genbank accession, protein, P08473 (Homo sapiens) (SEQ ID NO:4). The term “Neprilysin (NEP)”” also encompasses species variants, homologues, allelic forms, mutant forms, and equivalents thereof. In the methods described herein, fragments of these proteins can be detected. Neprilysin CD10 is a 100 kD neutral endopeptidase and a member of the metalloprotease family. It is a type II transmembrane protein, also known as common acute lymphoblastic leukemia antigen (CALLA), enkephalinase, neutral endopeptidase (NEP), membrane metallo-endopeptidase (MME) and CD10. Neprilysin is expressed on B cell precursors, T cell precursors, and neutrophils. Neprilysin is involved in B cell development and has been shown to bind opioid enkephalins, bradykinin, angiotensins I and II, and other biologically active peptides.

Commercial antibodies against neprilysin are available from ABCAM® catalog No. ab47721. Commercial ELISA kits are available from R and D Systems, catalog No. DY1182 (Human Neprilysin DuoSe) and from USCNLIFE™ catalog No: E1440h (CALLA ELISA kit).

“Zinc-α-2-glycoprotein (ZA2G)” refers to the ZAG or ZA2G protein of Genbank accession NP_{—}001176, protein, (Homo sapiens) (SEQ ID NO:5). The term “ZA2G” also encompasses species variants, homologues, allelic forms, mutant forms, and equivalents thereof. In the methods described herein, fragments of these proteins can be detected. Zinc-alpha-2-glycoprotein (ZA2G) is found in body fluids such as serum, sweat, and seminal and breast cyst fluids. It is identical in amino acid sequence to tumor-derived lipid mobilizing factor (LMF), a protein associated with the dramatic loss of adipose body stores in cancer cachexia, and has been shown to stimulate lipolysis by adipocytes in vivo and in vitro. A role for ZAG has been proposed in the regulation of body weight, and age-dependent changes in genetically influenced obesity, and also it regulates melanin production by normal and malignant melanocytes. It has also recently been classified as a novel adipokine in that it is produced by both white and brown fat adipocytes and may act in a local autocrine fashion in the reduction of adiposity in cachexia.

Commercial antibodies against human ZAG2 are available from BioVendor Laboratory Medicine, Inc. catalog Nos. RD181093100 and RD184093100, and from R and D Systems, catalog Nos. AF4764 and BAF4764. Commercial sandwich ELISA kits for human ZAG2 are available from BioVendor Laboratory Medicine, Inc. catalog No. RD191093100R.

“ALP” refers to alkaline phosphatase (ALP) Genbank accession BAA88367, protein, (Homo sapiens) (SEQ ID NO:6). The term “ALP” also encompasses species variants, homologues, allelic forms, mutant forms, and equivalents thereof. In the methods described herein, fragments of these proteins can be detected. ALP is also known as tissue-non specific isozyme precursor, is related to, but distinct from, intestinal/ALPI, placental/ALPP, and placental-like/ALPPL2 Alkaline Phosphatases. ALPL is expressed at very high levels in undifferentiated human embryonic stem cells, embryonic carcinoma cells, and embryonic germ cells. Mutations in this enzyme have been linked directly to hypophosphatasia, a disorder that is characterized by hypercalcemia and skeletal defects.

Commercial antibodies against human ALP are available from R and D Systems catalog Nos. MAB2909, FAB1448A, BAM1448, MAB1448, FAB1448P, and FAB1448C. Commercial ELISA kits for human ALP are available from ANASPEC Inc., SENSOLYTE™ pNPP Alkaline Phosphatase ELISA Assay Kit Colorimetric catalog No.71232-R and SENSOLYTE™ FDP Alkaline Phosphatase ELISA Assay Kit Fluorimetric catalog No.71101-R.

### Measuring Levels of Biomarker Protein

The levels of biomarker protein can be measured by any means known to those skilled in the art. In the present invention, it is generally preferred to use antibodies, or antibody equivalents, to detect levels of biomarker protein. However, other methods for detection of biomarker expression can also be used. Biomarker protein activity, can also be measured, for example peptidase activity can be measured.

In one embodiment, levels of biomarker protein are measured by contacting the biological sample with an antibody-based binding moiety that specifically binds to the biomarker protein, or to a fragment of the biomarker protein. Formation of the antibody-biomarker protein complex is then detected as a measure of biomarker protein levels.

The term “antibody-based binding moiety” or “antibody” includes immunoglobulin molecules and immunologically active determinants of immunoglobulin molecules, e.g., molecules that contain an antigen binding site which specifically binds (immunoreacts with) to the biomarker protein. The term “antibody-based binding moiety” is intended to include whole antibodies, e.g., of any isotype (IgG, IgA, IgM, IgE, etc), and includes fragments thereof which are also specifically reactive with biomarker protein. Antibodies can be fragmented using conventional techniques. Thus, the term includes segments of proteolytically-cleaved or recombinantly-prepared portions of an antibody molecule that are capable of selectively reacting with a certain protein. Non limiting examples of such proteolytic and/or recombinant fragments include Fab, F(ab′)₂, Fab′, Fv, dAbs and single chain antibodies (scFv) containing a VL and VH domain joined by a peptide linker. The scFv's may be covalently or non-covalently linked to form antibodies having two or more binding sites. Thus, “antibody-base binding moiety” includes polyclonal, monoclonal, or other purified preparations of antibodies and recombinant antibodies. The term “antibody-base binding moiety” is further intended to include humanized antibodies, bispecific antibodies, and chimeric molecules having at least one antigen binding determinant derived from an antibody molecule. In a preferred embodiment, the antibody-based binding moiety detectably labeled.

“Labeled antibody”, as used herein, includes antibodies that are labeled by a detectable means and include, but are not limited to, antibodies that are enzymatically, radioactively, fluorescently, and chemiluminescently labeled. Antibodies can also be labeled with a detectable tag, such as c-Myc, HA, VSV-G, HSV, FLAG, V5, or HIS.

In the diagnostic methods of the invention that use antibody-based binding moieties for the detection of biomarker levels, the level of biomarker present in the biological samples correlate to the intensity of the signal emitted from the detectably labeled antibody.

In one embodiment, the antibody-based binding moiety is detectably labeled by linking the antibody to an enzyme. The enzyme, in turn, when exposed to it's substrate, will react with the substrate in such a manner as to produce a chemical moiety which can be detected, for example, by spectrophotometric, fluorometric or by visual means. Enzymes which can be used to detectably label the antibodies of the present invention include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase. Chemiluminescence is another method that can be used to detect an antibody-based binding moiety.

Detection may also be accomplished using any of a variety of other immunoassays. For example, by radioactively labeling an antibody, it is possible to detect the antibody through the use of radioimmune assays. The radioactive isotope can be detected by such means as the use of a gamma counter or a scintillation counter or by audoradiography. Isotopes which are particularly useful for the purpose of the present invention are ³H, ¹³¹I, ³⁵S, ¹⁴C, and preferably ¹²⁵I.

It is also possible to label an antibody with a fluorescent compound. When the fluorescently labeled antibody is exposed to light of the proper wave length, its presence can then be detected due to fluorescence. Among the most commonly used fluorescent labeling compounds are CYE dyes, fluorescein isothiocyanate, rhodamine, phycoerytherin, phycocyanin, allophycocyanin, o-phthaldehyde and fluorescamine.

An antibody can also be detectably labeled using fluorescence emitting metals such as ¹⁵²Eu, or others of the lanthanide series. These metals can be attached to the antibody using such metal chelating groups as diethylenetriaminepentaacetic acid (DTPA) or ethylenediaminetetraacetic acid (EDTA).

An antibody also can be detectably labeled by coupling it to a chemiluminescent compound. The presence of the chemiluminescent-antibody is then determined by detecting the presence of luminescence that arises during the course of a chemical reaction. Examples of particularly useful chemiluminescent labeling compounds are luminol, luciferin, isoluminol, theromatic acridinium ester, imidazole, acridinium salt and oxalate ester.

In one preferred embodiment the biomarker proteins are detected by immunoassays, such as enzyme linked immunoabsorbant assay (ELISA), radioimmunoassay (RIA), Immunoradiometric assay (IRMA), or Western blotting, these assays are well known to those skilled in the art. Immunoassays such as ELISA or RIA, which can be extremely rapid, are more generally preferred. Antibody arrays or protein chips can also be employed, see for example U.S. Patent Application Nos: 20030013208A1; 20020155493A1; 20030017515 and U.S. Pat. Nos. 6,329,209; 6,365,418, which are herein incorporated by reference in their entirety.

The most common enzyme immunoassay is the “Enzyme-Linked Immunosorbent Assay (ELISA).” ELISA is a technique for detecting and measuring the concentration of an antigen using a labeled (e.g. enzyme linked) form of the antibody. There are different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. 1984, J. Clin. Chem. Clin. Biochem., 22:895-904.

In a “sandwich ELISA”, an antibody (e.g. anti-THP Uromodulin, anti-CD13 Aminopeptidase N (AMPN), anti-CD26 Dipeptidylpeptidase IV, anti-CD10 anti-Neprilysin (NEP), anti-ZAG ZA2G (zinc-α-2-glycoprotein) or anti-ALP) is linked to a solid phase (i.e. a microtiter plate) and exposed to a biological sample containing antigen (e.g. biomarker protein). The solid phase is then washed to remove unbound antigen. A labeled antibody (e.g. enzyme linked) is then bound to the bound-antigen (if present) forming an antibody-antigen-antibody sandwich. Examples of enzymes that can be linked to the antibody are alkaline phosphatase, horseradish peroxidase, luciferase, urease, and B-galactosidase. The enzyme linked antibody reacts with a substrate to generate a colored reaction product that can be measured.

In a “competitive ELISA”, antibody is incubated with a sample containing the biomarker protein (i.e. antigen). The antigen-antibody mixture is then contacted with a solid phase (e.g. a microtiter plate) that is coated with antigen. The more antigen present in the sample, the less free antibody that will be available to bind to the solid phase. A labeled (e.g., enzyme linked) secondary antibody is then added to the solid phase to determine the amount of primary antibody bound to the solid phase.

Other techniques may be used to detect the biomarkers of the invention, according to a practitioner's preference, and based upon the present disclosure. One such technique is Western blotting (Towbin et at., Proc. Nat. Acad. Sci. 76:4350 (1979)), wherein a suitably treated sample is run on an SDS-PAGE gel before being transferred to a solid support, such as a nitrocellulose filter. Detectably labeled antibodies that specifically bind to biomarker proteins can then be used to assess biomarker levels, where the intensity of the signal from the detectable label corresponds to the amount of biomarker present. Levels can be quantitated, for example by densitometry.

### Mass Spectrometry

In addition, biomarkers of the invention may be detected using Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos: 20030199001, 20030134304, 20030077616, which are herein incorporated by reference.

Mass spectrometry methods are well known in the art and have been used to quantify and/or identify biomolecules, such as proteins (see, e.g., Li et al. (2000) Tibtech 18:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000).

In certain embodiments, a gas phase ion spectrophotometer is used. In other embodiments, laser-desorption/ionization mass spectrometry is used to analyze the sample. Modern laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. However, MALDI has limitations as an analytical tool. It does not provide means for fractionating the sample, and the matrix material can interfere with detection, especially for low molecular weight analytes. See, e.g., U.S. Pat. No. 5,118,937 (Hillenkamp et al.), and U.S. Pat. No. 5,045,694 (Beavis & Chait).

In SELDI, the substrate surface is modified so that it is an active participant in the desorption process. In one variant, the surface is derivatized with adsorbent and/or capture reagents that selectively bind the protein of interest. In another variant, the surface is derivatized with energy absorbing molecules that are not desorbed when struck with the laser. In another variant, the surface is derivatized with molecules that bind the protein of interest and that contain a photolytic bond that is broken upon application of the laser. In each of these methods, the derivatizing agent generally is localized to a specific location on the substrate surface where the sample is applied. See, e.g., U.S. Pat. No. 5,719,060 and WO 98/59361. The two methods can be combined by, for example, using a SELDI affinity surface to capture an analyte and adding matrix-containing liquid to the captured analyte to provide the energy absorbing material.

For additional information regarding mass spectrometers, see, e.g., Principles of Instrumental Analysis, 3rd edition., Skoog, Saunders College Publishing, Philadelphia, 1985; and Kirk-Othmer Encyclopedia of Chemical Technology, 4.sup.th ed. Vol. 15 (John Wiley & Sons, New York 1995), pp. 1071-1094.

Detection of the presence of a marker or other substances will typically involve detection of signal intensity. This, in turn, can reflect the quantity and character of a polypeptide bound to the substrate. For example, in certain embodiments, the signal strength of peak values from spectra of a first sample and a second sample can be compared (e.g., visually, by computer analysis etc.), to determine the relative amounts of particular biomolecules. Software programs such as the Biomarker Wizard program (Ciphergen Biosystems, Inc., Fremont, Calif.) can be used to aid in analyzing mass spectra. The mass spectrometers and their techniques are well known to those of skill in the art.

Any person skilled in the art understands, any of the components of a mass spectrometer (e.g., desorption source, mass analyzer, detect, etc.) and varied sample preparations can be combined with other suitable components or preparations described herein, or to those known in the art. For example, in some embodiments a control sample may contain heavy atoms (e.g. ¹³C) thereby permitting the test sample to mixed with the known control sample in the same mass spectrometry run.

In one preferred embodiment, a laser desorption time-of-flight (TOF) mass spectrometer is used. In laser desorption mass spectrometry, a substrate with a bound marker is introduced into an inlet system. The marker is desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of molecules of specific mass to charge ratio.

In some embodiments the relative amounts of one or more biomolecules present in a first or second sample is determined, in part, by executing an algorithm with a programmable digital computer. The algorithm identifies at least one peak value in the first mass spectrum and the second mass spectrum. The algorithm then compares the signal strength of the peak value of the first mass spectrum to the signal strength of the peak value of the second mass spectrum of the mass spectrum. The relative signal strengths are an indication of the amount of the biomolecule that is present in the first and second samples. A standard containing a known amount of a biomolecule can be analyzed as the second sample to provide better quantify the amount of the biomolecule present in the first sample. In certain embodiments, the identity of the biomolecules in the first and second sample can also be determined.

In one preferred embodiment, biomarker levels are measured by MALDI-TOF mass spectrometry.

Detection of biomarker proteins using activity assays are also contemplated. Such methods are known in the art, e. g. the radiochemical assay described by Ryan J W, in Anal. Biochem. 1993 Apr;210(1):27-33.

### Antibodies

The antibodies for use in the present invention can be obtained from a commercial source. Alternatively, antibodies can be raised against the biomarker protein to be detected, or a portion of the biomarker polypeptide. Methods useful for the production of antibodies are disclosed in U.S. Application. Nos. 2002/0182702; 2003/0212256; 20020110894 and WO 01/11074, which are herein incorporated by reference.

Antibodies for use in the present invention can be produced using standard methods to produce antibodies, for example, by monoclonal antibody production (Campbell, A. M., Monoclonal Antibodies Technology: Laboratory Techniques in Biochemistry and Molecular Biology, Elsevier Science Publishers, Amsterdam, the Netherlands (1984); St. Groth et al., J. Immunology, (1990) 35: 1-21; and Kozbor et al., Immunology Today (1983) 4:72). Antibodies can also be readily obtained by using antigenic portions of the protein to screen an antibody library, such as a phage display library by methods well known in the art. For example, U.S. Pat. No. 5,702,892 (U.S.A. Health & Human Services) and WO 01/18058 (Novopharm Biotech Inc.) disclose bacteriophage display libraries and selection methods for producing antibody binding domain fragments.

### Detection Kits

The present invention is also directed to commercial kits for the detection and prognostic evaluation of CP/CPPS. The kit can be in any configuration well known to those of ordinary skill in the art and is useful for performing one or more of the methods described herein for the detection of biomarker proteins. The kits are convenient in that they supply many if not all of the essential reagents for conducting an assay for the detection of biomarker protein in a urine sample, pre and post-prostatic massage. In addition, the assay is preferably performed simultaneously with a standard or multiple standards that are included in the kit, such as a predetermined amount of biomarker protein, so that the results of the test can be quantitated or validated.

The kits include an assay means for detecting biomarker levels such as antibodies, or antibody fragments, which selectively bind to biomarker protein In one embodiment, the kits provide at least one antibody-based binding moiety that binds to biomarker protein uromodulin (THP), Aminopeptidase N (AMPN), dipeptidylpeptidase IV (CD26), neprilysin (NEP), zinc-α-2-glycoprotein (ZA2G) and alkaline phosphatase (ALP) and a suitable container means. It is contemplated that in particular embodiments, the kit can further comprise a second antibody preparation (preferably detectably labeled) that binds immunologically to the same biomarker protein as the first antibody preparation, but where the first and the second antibodies bind to different epitopes; and a suitable container means thereof. In a particularly preferred aspects the first antibody preparation is attached to a support. It is contemplated that the support can be any support routinely used in immunological techniques. Any solid support can be used, including but not limited to, nitrocellulose, solid organic polymers, such as polystyrene, solid support beads or laminated dipsticks such as described in U.S. Pat. No. 5,550,375. In a particularly preferred embodiments, the support independently is a polystyrene plate, test tube or dipstick. One preferred embodiment includes the use of a dip-stick.

The use of “dip sticks” or test strips and other solid supports have been described in the art in the context of an immunoassay for a number of antigens. Three U.S. patents (U.S. Pat. No. 4,444,880, issued to H. Tom; U.S. Pat. No. 4,305,924, issued to R. N. Piasio; and U.S. Pat. No. 4,135,884, issued to J. T. Shen) describe the use of “dip stick” technology to detect soluble antigens via immunochemical assays. The apparatuses and methods of these three patents broadly describe a first component fixed to a solid surface on a “dip stick” which is exposed to a solution containing a soluble antigen that binds to the component fixed upon the “dip stick,” prior to detection of the component-antigen complex upon the stick.

The kits can include multiple antibodies that interact with each of the biomarker proteins of the invention, such that multiple biomarker proteins can be measured.

Accordingly, in one embodiment, a kit designed for facilitating the diagnosis of chronic prostatitis/chronic pain pelvic syndrome (CP/CPPS) in a subject comprises a means for detecting, in urine, one or more protein biomarkers selected from the group consisting of uromodulin (THP), Aminopeptidase N (AMPN), dipeptidylpeptidase IV (CD26), neprilysin (NEP), zinc-α-2-glycoprotein (ZA2G) and alkaline phosphatase (ALP); a container for holding a urine sample; and instructions for use, wherein the means for detecting one or more of the protein biomarkers comprises an antibody-based binding moiety that specifically binds to the biomarker to be detected.

In some embodiments, the kit comprises at least two, at least three, at least 4, at least five, or at least 6 antibody-based binding moieties which detect the presence or levels of at least two, three, four, five or six protein biomarkers.

In some embodiments, the kit comprises at least two antibodies, one antibody is immobilized on a solid phase and one antibody is detectably labeled.

In other embodiments, the assay kits to measure the level of biomarker protein employ (but are not limited to) the following techniques: competitive and non-competitive assays, radioimmunoassay (RIA), bioluminescence and chemiluminescence assays, fluorometric assays, sandwich assays, immunoradiometric assays, dot blots, enzyme linked assays including ELISA, microtiter plates, and immunocytochemistry. For each kit the range, sensitivity, precision, reliability, specificity and reproducibility of the assay are established by means well known to those skilled in the art.

The above described assay kits would further provide instructions for use.

The present invention can be defined by any of the following alphabetized paragraphs:
[A] A kit designed for facilitating the diagnosis of chronic prostatitis/chronic pelvic pain syndrome (CP/CPPS) in a human comprising:
   at least one solid support;
   at least three antibody-based moieties specific for each of three different biomarker proteins, wherein the three biomarker proteins comprises uromodulin (THP), aminopeptidase N (AMPN), and neprilysin (NEP);
   at least three labeled antibody-based moieties specific for each of the three different biomarker proteins; and
   instruction for use and interpretation of the kit.
[B] The kit of paragraph [A], further comprising (a) a fourth antibody-based moiety specific for a fourth biomarker protein, wherein the fourth biomarker protein is selected from the group consisting of dipeptidylpeptidase (CD26), zinc-α-2-glycoprotein (ZA2G) and alkaline phosphatase (ALP); and (b) a fourth labeled antibody-based moiety specifically binding the fourth biomarker protein.
[C] The kit of paragraph [B], further comprising (a) a fifth antibody-based moiety specific for a fifth biomarker protein, wherein the fifth biomarker protein is selected from the group consisting of dipeptidylpeptidase (CD26), zinc-α-2-glycoprotein (ZA2G) and alkaline phosphatase (ALP); and (b) a fifth labeled antibody-based moiety specifically binding the fifth biomarker protein.
[D] The kit of paragraph [C], further comprising (a) a sixth antibody-based moiety specific for a sixth biomarker protein, wherein the sixth biomarker protein is selected from the group consisting of dipeptidylpeptidase (CD26), zinc-α-2-glycoprotein (ZA2G) and alkaline phosphatase (ALP); wherein the fourth, fifth and sixth biomarker proteins are different; and (b) a sixth labeled antibody-based moiety specific for the sixth biomarker protein.
[E] The kit of any of paragraphs [A]-[D], wherein an antibody-based moiety: biomarker protein complex is formed when the solid support is contacted with the sample.
[F] The kit of any of paragraphs [A]-[D], further comprises standards of known amounts of said biomarker proteins.
[G] The kit of any of paragraphs [A]-[D], further comprises reference values of the levels of biomarker proteins.
[H] The kit of paragraphs [G], wherein the reference values are average levels of each of the biomarker protein in samples from a population of healthy male humans not having CP/CPPS.
[I] The kit of any of paragraphs [A]-[D], wherein at least one of said antibody-based moieties is immobilized on at least one solid support.
[J] The kit of any of paragraphs [A]-[D], wherein at least one of said labeled antibody-based moieties is immobilized on at least one solid support.
[K] The kit of paragraphs [I] or [J], wherein each of said antibody-based moiety is immobilized on the same solid support.
[L] The kit of paragraphs [I] or [J], wherein at least one of said antibody-based moieties is immobilized on a first solid support and at least another of said antibody-based moieties is immobilized on a second solid support.
[M] The kit of any of paragraphs [A]-[D], wherein the labeled antibody-based moieties are detectably labeled and detectable label is selected from a group consisting of enzyme, fluorescent, biotin, gold, latex and radioisotope labeling.
[N] The kit of any of paragraphs [A]-[D], wherein at least one of said labeled antibodies comprises an enzyme-labeled antibody.
[O] The kit of any of paragraphs [A]-[D], further comprising at least one sample collection container for sample collection.
[P] The kit of any of paragraphs [A]-[D], further comprising instructions for use and interpretation of results.
[Q] The kit of any of paragraphs [A]-[D], comprising a test strip or a dipstick.
[R] A method for facilitating the diagnosis of a subject for chronic prostatitis/chronic pain pelvic syndrome (CP/CPPS) comprising:
   obtaining a post-prostatic massage urine sample from the subject; and
   detecting the presence of at least one biomarker protein in the post-prostatic massage urine sample, wherein the biomarker protein is selected form the group consisting of uromodulin (THP), aminopeptidase N (AMPN), dipeptidylpeptidase IV (CD26), neprilysin (NEP), zinc-α-2-glycoprotein (ZA2G) and alkaline phosphatase (ALP); and
   wherein the presence of at least one biomarker protein is indicative of the presence of CP/CPPS.
[S] The method of paragraph [R], wherein the post-prostatic massage sample is compared to a control sample, wherein the control sample is a post-prostatic message urine sample from a subject that is asymptomatic for CP/CPPS.
[T] The method of paragraph [R], wherein the presence of at least two biomarkers is indicative of the presence of CP/CPPS.
[U] The method of paragraph [R], wherein the presence of at least three biomarkers is indicative of the presence of CP/CPPS.
[V] The method of paragraph [R], wherein the presence of at least four biomarkers is indicative of the presence of CP/CPPS.
[W] The method of paragraph [R], wherein the presence of at least five biomarkers is indicative of the presence of CP/CPPS.
[X] The method of paragraph [R], wherein the presence of at least six biomarkers is indicative of the presence of CP/CPPS.
[Y] A method for facilitating the diagnosis of a subject for chronic prostatitis/chronic pain pelvic syndrome (CP/CPPS) comprising:
   measuring the level of at least one biomarker protein in a post-prostatic massage urine test sample obtained from the patient; and
   comparing the level of the biomarker measured in the test sample with the level of the same biomarker protein present in a control sample;
   wherein a higher level of the measured biomarker protein in the test sample as compared to the level of the same biomarker protein in the control sample is indicative of CP/CPPS, and wherein the biomarker protein is selected form the group consisting of uromodulin (THP), aminopeptidase N (AMPN), dipeptidylpeptidase IV (CD26), neprilysin (NEP), zinc-α-2-glycoprotein (ZA2G) and alkaline phosphatase (ALP).
[Z] The method of paragraph [Y], wherein the presence of the biomarker protein is detected using an antibody-based moiety which specifically binds to the biomarker protein.
[AA] The method of paragraph [Y], wherein the levels of at least 2, 3, 4, 5, or 6 biomarker proteins are measured and higher levels of at least 2, 3, 4, 5, or 6 biomarker proteins in the test sample as compared to the levels of biomarker proteins in the control sample is indicative of CP/CPPS
[BB] The method of paragraph [Y], wherein the level of the biomarker protein is measured by measuring the activity of the biomarker.
[CC] The method of paragraph [Y], wherein the level of biomarker protein is measured by a method comprising the steps of:
   contacting the test sample, or preparation thereof, with an antibody-based binding moiety which specifically binds the biomarker protein to form an antibody-biomarker protein complex; and
   detecting the presence of the complex, thereby measuring the level of biomarker protein present.
[DD] The method according to paragraph [Z] or [CC], wherein the antibody-based binding moiety is labeled with a detectable label.
[EE] The method according to paragraph [DD], wherein the label is selected from the group consisting of a radioactive label, a hapten label, a fluorescent label, and an enzymatic label.
[FF] The method according to paragraph [Z] or [CC], wherein the antibody-based binding moiety is an antibody.
[GG] The method according to paragraph [FF], wherein the antibody is an monoclonal antibody.

The references cited throughout the specification are hereby incorporated by reference.

The present invention is further illustrated by the following Examples. These Examples are provided to aid in the understanding of the invention and are not construed as a limitation thereof.

### EXAMPLE 1

### Identification of Biomarkers for CP/CPPS in Post-Prostatic Massage Urine

Chronic prostatitis/chronic pelvic pain syndrome (CP/CPPS)—a symptom complex of unknown etiology manifested by pain or discomfort in the pelvic region for at least 3 months in the previous 6 months. CP/CPPS is a frustrating clinical syndrome that lacks pathognomonic biomarkers. The objective of the present study was to examine the pre- and post-prostatic massage (pre-M and post-M) urine of CP/CPPS patients and controls to discover potential diagnostic biomarkers for CP/CPPS.

Thirty CP/CPPS patients and 30 healthy asymptomatic age-matched controls were enrolled in the study. Inclusion and exclusion criteria were those recommended by the Chronic Prostatitis Collaborative Research Network (CPCRN). Controls were defined as healthy asymptomatic men drawn from the general population with an NIH-CPSI score of zero, a PSA level of less than 2.5 ng/mL and a negative digital rectal exam for prostate cancer. The study included one clinical visit during which pre-massage and post-massage urine samples and the NIH-CPSI were collected. All subjects provided written informed consent and the study protocol was approved by the IRB at each institution providing samples. Protein identification was conducted at two independent sites, the Center for Applied Proteomics and Molecular Medicine at George Mason University and the Taplin Mass Spectrometry Center at Harvard Medical School. Urine proteins were separated by SDS-PAGE (4-20%) and proteins were visualized by silver or Coomassie Blue (GelCode Blue) staining. Protein spots were excised from the gel and in-gel digested with trypsin (Promega, Madison, Wis.) according to published protocols. The significance of the difference in up-regulation between CP/CPPS cases and controls was evaluated using the Fisher's exact test.

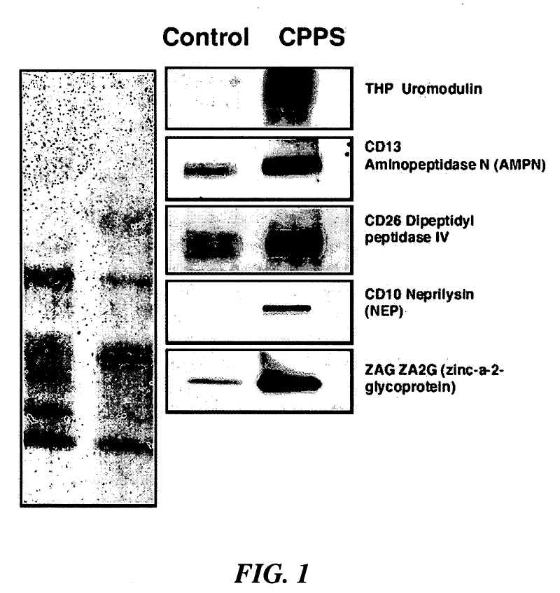

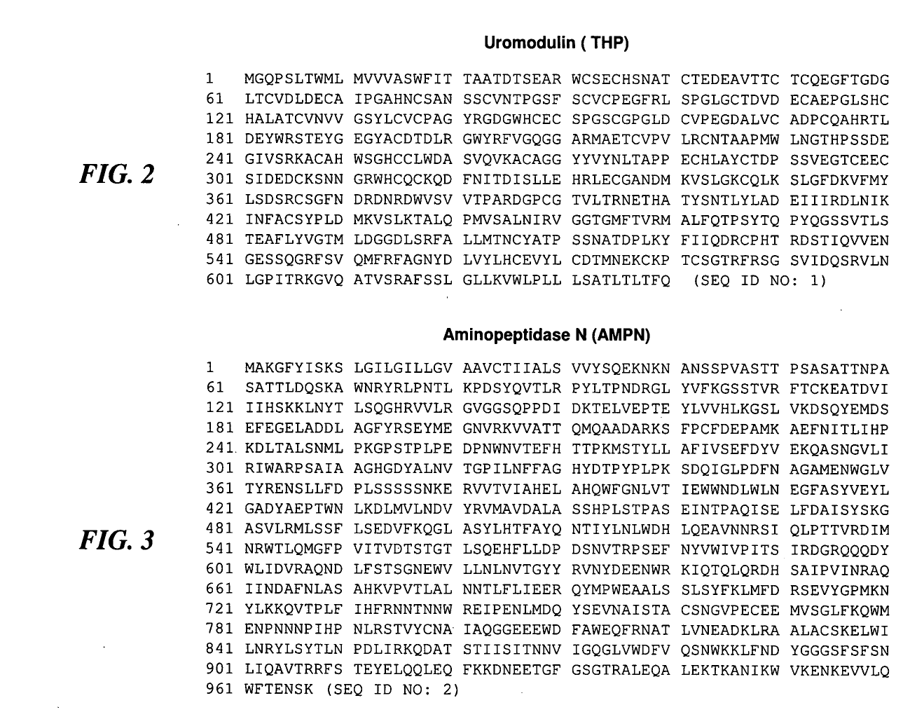

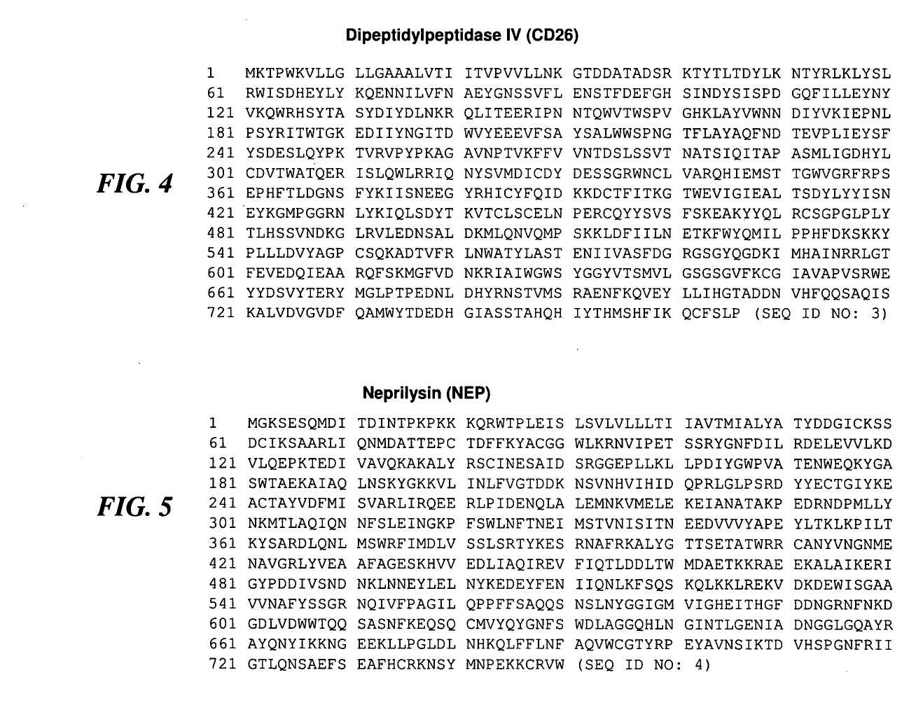

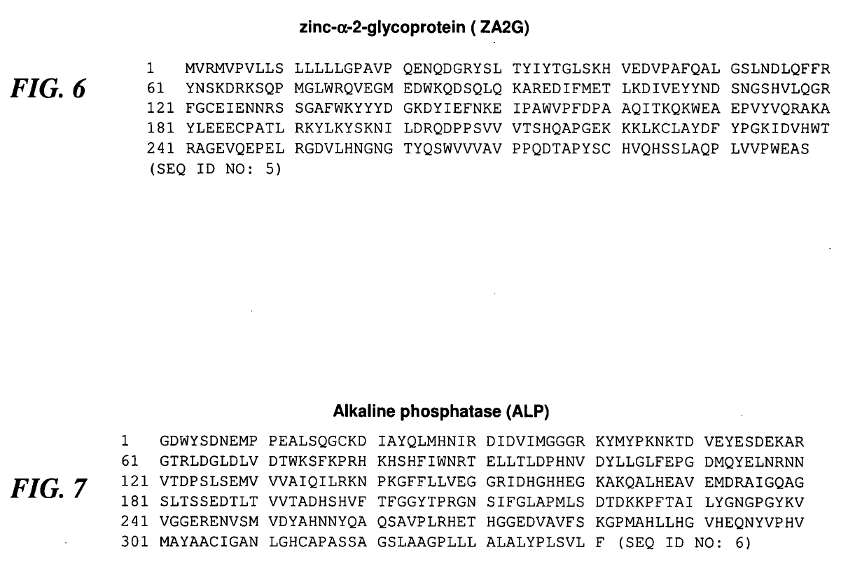

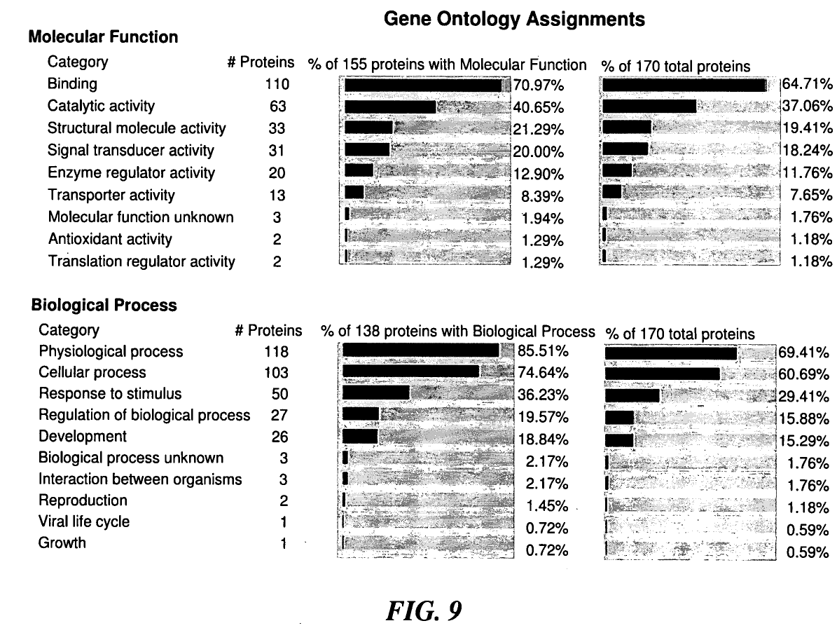

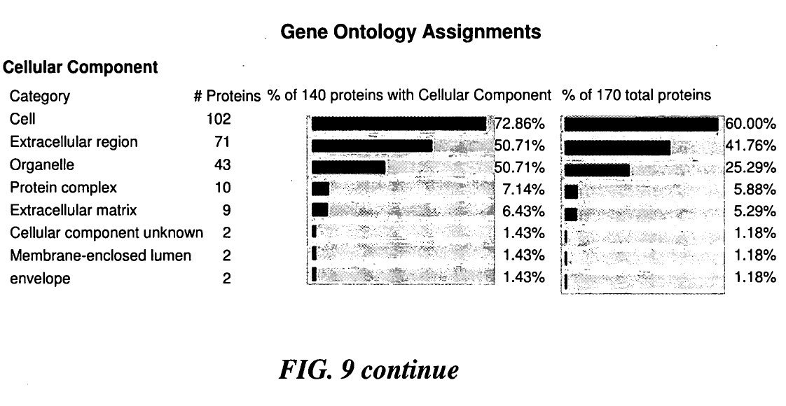

A total of 177 unique proteins was identified in the post-M of men with CP/CPPS. EASE analysis using GO terms and KEGG pathways revealed several pathways that were up-regulated in the post-M CP/CPPS protein set. The most significant GO terms found (P<0.001) and the most significant KEGG pathways (P<0.1) are shown in FIG. 9. Six proteins—alkaline phosphatase (ALP), zinc-α-2-glycoprotein (ZA2G), CD10 Neprilysisn (NEP), APN (CD13 Aminopeptidase N; AMPN), DPP (CD26 Dipeptidyl peptidase IV), and THP Uromodulin—were reconfirmed with Western Blot analysis (FIG. 1) and dot blot analysis (FIG. 8).

Conclusion: The post-M of men with CP/CPPS shows a specific proteomic signature that contains several potential diagnostic biomarkers for CP/CPPS. Using a proteomics approach we have identified six biomarkers that are uniquely expressed in chronic prostatitis/chronic pain syndrome (CP/CPPS). The biomarkers were identified to be THP/Uromodulin, CD13/Aminopeptidase N (AMPN; APN), CD26/Dipeptidylpeptidase IV, CD10/Neprilysin (NEP), ZA2G (zinc-α-2-glycoprotein) and alkaline phosphatase (ALP). These six protein biomarkers were confirmed using Western blot analysis, a representative Western Blot is shown in FIG. 1. THP/Uromodulin, CD13/Aminopeptidase N (AMPN), CD26/Dipeptidylpeptidase IV, CD10/Neprilysin (NEP), ZA2G (zinc-α-2-glycoprotein) and alkaline phosphatase (ALP) are present at significantly higher levels in post-prostatic massage urine samples from CP/CPPS patients as compared to control post-prostatic massage urine samples from patients that are asymptomatic for CP/CPPS.

### Example 2

### Diagnostic Test Strips-Design 1

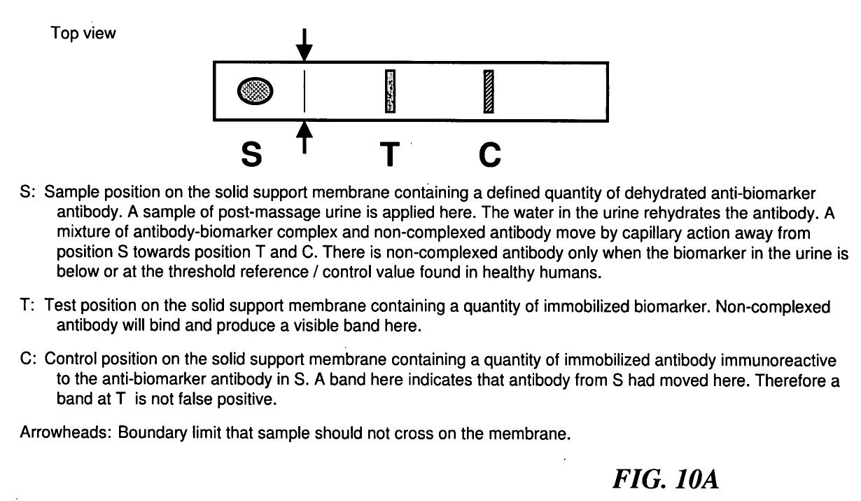

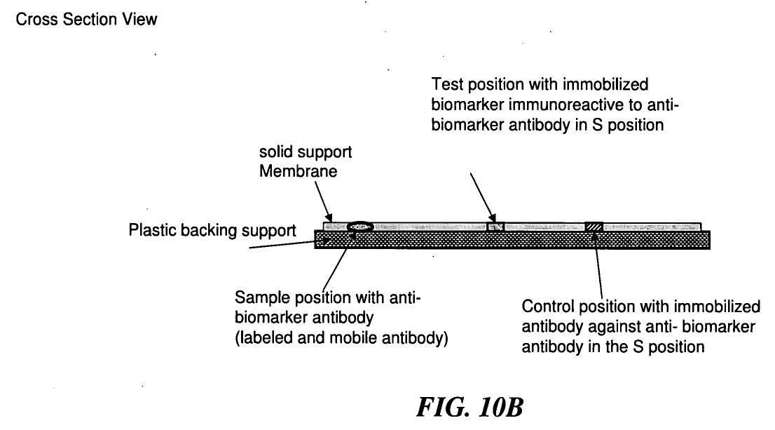

The levels of biomarker proteins described herein can be determined using test strips as illustrated in FIG. 10-13. In the test strip, the membrane is divided into three separate regions: a sample (S) position at one end of the membrane, a test (T) position located at the middle of the membrane, and a control (C) position found at the opposite end the membrane (FIG. 10A). Located at S is a defined quantity of dehydrated anti-biomarker protein antibody. The antibody can be conjugated to colloidal gold beads or colored latex beads for visualization purposes. At T, there is a defined quantity of biomarker protein immobilized on the membrane. At C, there is another immobilized protein, an antibody immunoreactive to the anti-biomarker protein antibody located at the S position (FIG. 10).

The defined quantity of dehydrated anti-protein antibody at S position is such that there is just enough antibody to bind the biomarker protein from the sample (e. g. urine) when the protein is at the reference/control level. The reference/control level can be the level of the biomarker found in the samples of healthy men. Therefore, when the biomarker protein is at or above the reference level, all the anti-biomarker antibody at the S position will be bound to the protein in the form of protein-antibody complex; there will be no free anti-biomarker protein antibody present.

The choice of the anti-biomarker protein antibody placed at the S position can be any antibody that is specially immunoreactive to any of the proteins of interest. The antibody can be monoclonal, polyclonal, or a mixture of both monoclonal and polyclonal antibodies. Antibody-based moiety can also be used. When only one protein is studied, the S position will have only one antibody specially immunoreactive with just that one protein of interest. On the other hand if three proteins are to be studied simultaneously, the S position will have three different types of anti-protein antibodies, each type specifically immunoreactive to one protein and does not exhibit cross-reactivity with the other two non-ligand proteins (FIG. 13).

The S position is where a sample (e. g. urine) is applied. The arrowheads delineate the boundary limit that the sample should not cross on the membrane when applying the sample to the membrane.

The defined quantity of protein immobilized on the membrane at the T position (FIG. 10) is a quantity that will bind and capture any free anti-protein antibody from the S position that is not found in a protein-antibody complex form. That quantity of immobilized protein will serve to immobilize free anti-protein antibody to the T position during testing.

When only one protein is studied, only one protein, the same type as the one of interest will be immobilized at the T position. Likewise when three proteins are to be studied simultaneously, all three protein types will be represented at the T position and at their respective quantities (FIG. 13).

The antibody at the C position can be a monoclonal, polyclonal, or a mixture of both monoclonal and polyclonal antibodies. Antibody-based moiety can also be used. When more than one protein is studied simultaneously, there will a corresponding number of anti-S-position antibodies and these antibodies should be specific for their respective ligand, the S position anti-protein antibody, and should not cross-react with non-ligand (FIG. 13).

The application of a sample (e. g. urine) at the S position will re-hydrate the anti-biomarker protein antibody and result in the antibody-protein complex formation. By capillary action along the membrane, the fluid mixture of antibody and protein will move toward the T position and subsequently to the C position. When the protein of interest is below the reference level, the mixture of antibody and protein will contain free anti-protein antibody and antibody-protein conjugates/complexes. Upon arrival at the T position, the free anti-biomarker protein antibody will bind the immobilized protein and be immobilized at the T position. The localized concentration of free anti-protein antibody that is colloidal gold or latex bead labeled will become visible as a colored line at the T position (FIG. 11, middle). When the protein of interest is at or above the reference level, the mixture of antibody and protein will contain all antibody-protein complexes and no free anti-protein antibody. At the T position, there will be no colloidal gold or latex bead labeled anti-protein antibody, and the area remains clear (FIG. 11, left). At the C position the protein-antibody complexes will be bound and captured by the immobilized antibody immunoreactive against the anti-protein antibody coming from the S position. This will in turn results in a concentration of a colloidal gold or latex bead labeled anti-protein antibody at the C position and will become visible as colored line at the C position. The C position result serves as a test control that there is functional anti-protein antibody in the test material and should always be present (FIG. 11, right).

The test strip can be designed in a form of a dipstick test strip (FIG. 10B). As a dipstick test strip, the strip is dipped into a sample (e. g. urine) at the S position end with sample level not to exceed the boundary limit. The strip is then laid horizontally with the membrane surface facing up on a flat surface. A fixed amount of time is given for the antibody re-hydration, capillary action, and antibody binding reaction to take place. At the end of the fixed time, there should be visible bands at the C position and depending on the level of the protein of interest, there may or may not be a visible band at the T position (FIG. 11). FIG. 12 shows a method of using three separate dipstick test strips to test for the three proteins of interest. Each dipstick test strip is labeled to indicate which biomarker protein is being tested. A diagnostic kit can comprise multiple types of single biomarker test strips, a type for each biomarker of interest. FIG. 13 shows an alternative design where three proteins can be studied simultaneously on the same membrane and therefore on the same dipstick test strip. The positions of the expected results in the T and C positions for each protein are indicated.

### Example 3

### Test Strips-Design 2

An alternative version of the test strips for determining the level of biomarker protein level is illustrated in FIG. 14. Here the membrane strip contains two different anti-biomarker antibodies specific for the same biomarker, each antibody binds the biomarker at a different epitope. The first antibody is labeled (e. g. colored latex beads), disposed on the solid support membrane but is not immobilized on it, (i. e. the antibody is mobile), and is disposed in excess at the S position. The second anti-biomarker antibody is not labeled but is immobilized and is in excess at position T. This second anti-biomarker protein antibody binds an epitope on the biomarker that is not affected by the binding of the first antibody. At position C, there is an excess of non-labeled antibody against the anti-biomarker antibody at the S position. The antibody at C serves to capture any free labeled anti-biomarker antibody migrating from S. When sufficient free labeled anti-biomarker antibody is accumulated at C, a visible band appears. The band is a control to confirm that the band(s) observed on the test strip are due to the mobile antibody at the S position.

As described for the test strips in example 2, when a fluid sample (e. g. urine) is place at the S position, a mixture of free anti-biomarker antibody and biomarker: antibody complexes is formed. The mixture migrates by capillary action towards the T and the C positions. The second anti-biomarker antibody immobilized at T will capture all the biomarker:antibody complexes. Only when the biomarker is at or above the reference level will sufficient labeled antibody be captured at T to produce a visible band (FIG. 14). When the biomarker is below the reference level, no visible band should appear at the T position.

### Example 4

### Test Strips-Design 3

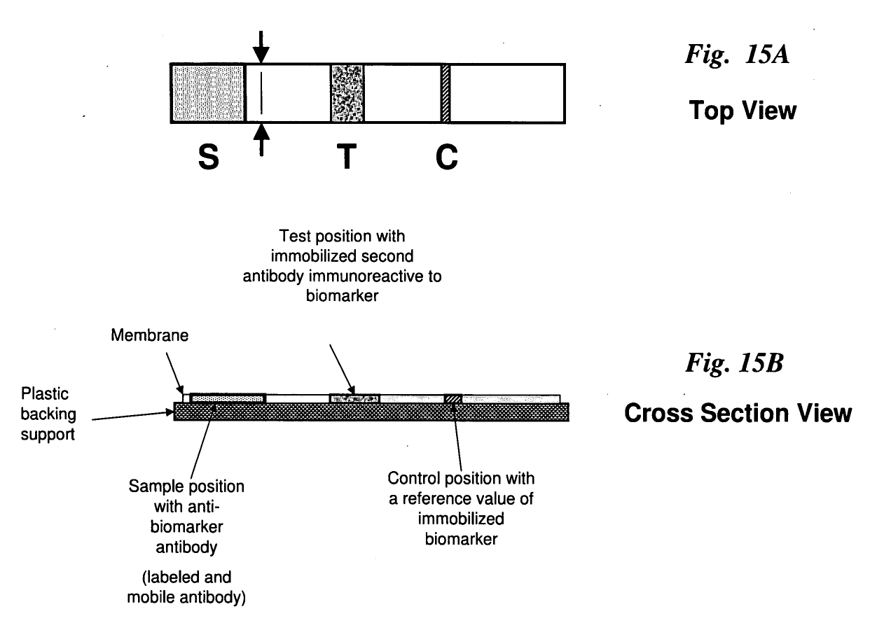

An alternative version of the test strips for determining the level of biomarker protein level is illustrated in FIG. 15. There is the same single protein-binding membrane strip as described in FIG. 10 that is divided into three separate regions: a sample (S), a test (T), and a control (C) positions, all three spatially arranged as shown in FIG. 10 and FIG. 15. In this design, the S position contain an excess amount of dehydrate anti-biomarker protein antibody that can be labeled (e. g. colloidal gold or color latex bead). Similar to the design in FIG. 10-13, the anti-biomarker antibody at S is mobile; once the antibody is re-hydrated, the antibody moves by capillary action towards the T and C position.

The T position contains a second antibody that is also immunoreactive to the biomarker protein of interest, but to a different epitope on the biomarker (FIG. 15). This second antibody is in excess and is immobilized on the membrane. This second anti-biomarker protein antibody binds a part of the biomarker protein that is different from the part of the protein that is bound by the first anti-biomarker protein antibody found at the S position. In this design, the second antibody at the T position will bind and capture both free unbound protein and protein-antibody conjugates, and concentrate them at the T position.

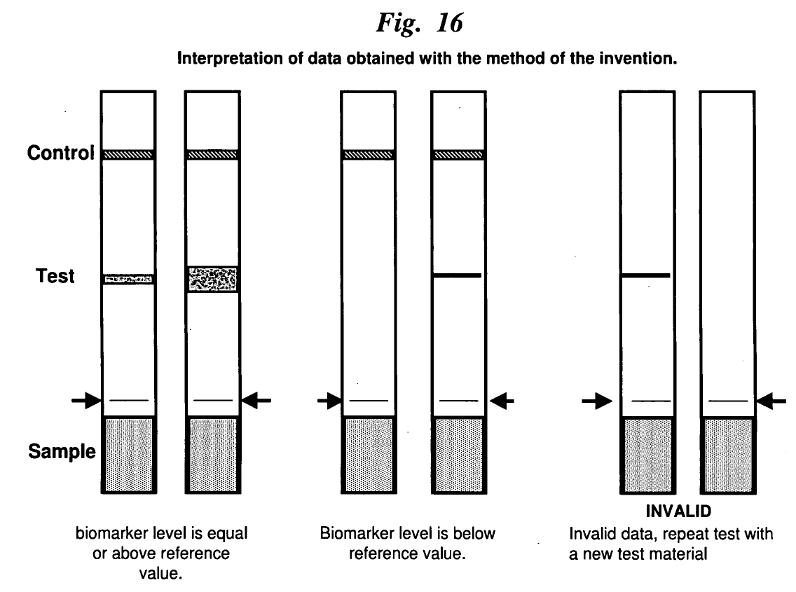

The C position contains a defined quantity of biomarker protein immobilized on the membrane (FIG. 15B). The defined quantity is the reference value of the biomarker protein under study. The reference/control level can be the level of the biomarker found in the samples of healthy men. When the excess free anti-biomarker protein from the S position arrives and bind the immobilized protein, gradually becomes concentrated at the C position and will become visible as a colored line at the C position (FIG. 16).

An application of a fluid sample (e. g. urine or serum) at the S position will re-hydrate the excess amount of anti-biomarker protein antibody there. A fluid mixture of free antibody and protein-antibody complex is formed and will move along the membrane by capillary action towards the T position and then subsequently to the C position. At the T position, all proteins will be bound and immobilized, gradually become concentrated and visible as a colored line. With increasing amount of protein bound and concentrated at the T position, the colored line expands and develop into a band. The greater the level of protein of interest in the sample, the wider the colored band at the T position (FIG. 16, left). When the excess free anti-protein antibody from the S position arrives to the C position and bind to the immobilized protein, another color line become visible. Since there is a reference amount of immobilized proteins at the C position, the thickness of the visible colored line at the C position defines the reference value of protein. By comparing the thickness of the color band at the T and C positions on the same membrane, one can estimate whether the serum protein level is below or greater than the reference value of the protein. When the sample protein level is equal or greater than the reference value, the color band at the T position will be equal or larger than the color band at the C position respectively (FIG. 16, left). When the serum protein level is below the threshold level, the color band at the T position will be smaller or even absent than the color band at the C position (FIG. 16, middle). The C position band also serves as a test control to confirm that there is functional anti-protein antibody at the S position and that the functional anti-protein antibody is derived from the S position (FIG. 16, right).

This immunological assay may be designed in the form of a dipstick test strip as shown in FIG. 17 where individual strip is used for each biomarker protein and the strips are labeled. A diagnostic kit can comprise multiple test strips, at least one for each of the biomarker of interest. Another design is a single test strip where three biomarker proteins can be studied simultaneously on the same membrane (FIG. 18). The positions of the expected results in the T and C positions for each protein are indicated.

### Example 5

### Diagnostic ELISA Assay Design 1

The levels of biomarker proteins described herein can be determined using an ELISA assay as illustrated in FIG. 19. An ELISA assay comprises performing an standard titration assay and a sample assay in order to determine the amount of protein present in the sample. As shown in FIG. 19, the ELISA assay microtiter plate consists of four reference rows: a reference row for each of the four biomarker proteins, aminopeptidase, neprilysin, uromodulin, and zinc-α-2-glycoprotein, and two test/sample rows. The reference rows and sample wells for the different protein of interest are labeled (FIG. 19). Excess amounts of anti-biomarker protein antibodies are immobilized in the wells of plate. There is specific antibody for each of the biomarker protein of interest. Standard amounts of protein ranging from 0-50 pg/ml, ng/ml, μg/ml, or mg/ml are placed in the reference rows to create a standard curve for each of the biomarker protein of interest. The serum sample is placed in the sample wells. Subsequently, a enzyme-labeled (e. g. horse-radish peroxidase) anti-biomarker protein antibody specific for each of the biomarker protein of interest is added to the wells. The mixtures in the wells are allowed to incubate at room temperature for 90 min and the liquid is decanted. The wells are washed five times with deionized water. The, an aliquot of 3, 3′, 5, 5′ tetramethylbenzidine (TMB) reagent is added into each well. The mixture is gently mixed for 10 seconds and incubate at room temperature (18-25° C.) for 20 minutes. The enzymatic reaction is terminated by adding IN HCl. Gentle agitation is carried out till all the blue color changes to yellow color completely. The amount of color by-product is determined by reading its absorbance at 450 nm with a microtiter well reader. The A450 correspond to the amount of biomarker protein in the well. The amount of the biomarker protein in a test sample can be estimated from the A450 obtained from the sample wells and the standard curve obtained from the reference wells.

### Example 6

### Diagnostic ELISA Assay Design 2

Alternatively, the second ELISA assay as shown in FIG. 20 can be used. As in FIG. 19, the reference rows and sample wells for the different biomarker protein of interest are labeled (FIG. 20). Excess amounts of anti-protein antibodies are immobilized in the wells of plate, with specific antibody for each of the protein of interest. A fixed amount of each of protein of interest is placed in duplicate reference wells. This fixed amount is the reference value corresponds to the average amount of the protein found in the samples of healthy men. The sample, e. g. urine, is also placed in the duplicate sample wells. The assay plate is process as described herein. The A450 obtained from the sample wells are compared with those obtained for the corresponding reference rows in order to determine whether there is an increase or decrease in the amount of the protein in the sample.

We claim

1. A kit designed for facilitating the diagnosis of chronic prostatitis/chronic pelvic pain syndrome (CP/CPPS) in a human comprising:
a. at least one solid support;
b. at least three antibody-based moieties specific for each of three different biomarker proteins, wherein said three biomarker proteins comprise uromodulin (THP), aminopeptidase N (AMPN), and neprilysin (NEP);
c. at least three labeled antibody-based moieties specific for each of the three different biomarker proteins; and
d. instruction for use and interpretation of the kit.

2. The kit of claim 1, further comprising (a) a fourth antibody-based moiety specific for a fourth biomarker protein, wherein the fourth biomarker protein is selected from the group consisting of dipeptidylpeptidase (CD26), zinc-α-2-glycoprotein (ZA2G) and alkaline phosphatase (ALP); and (b) a fourth labeled antibody-based moiety specifically binding said fourth biomarker protein.

3. The kit of claim 2, further comprising (a) a fifth antibody-based moiety specific for a fifth biomarker protein, wherein the fifth biomarker protein is selected from the group consisting of dipeptidylpeptidase (CD26), zinc-α-2-glycoprotein (ZA2G) and alkaline phosphatase (ALP); and (b) a fifth labeled antibody-based moiety specifically binding said fifth biomarker protein.

4. The kit of claim 3, further comprising (a) a sixth antibody-based moiety specific for a sixth biomarker protein, wherein the sixth biomarker protein is selected from the group consisting of dipeptidylpeptidase (CD26), zinc-α-2-glycoprotein (ZA2G) and alkaline phosphatase (ALP); wherein the fourth, fifth and sixth biomarker proteins are different; and (b) a sixth labeled antibody-based moiety specific for said sixth biomarker protein.

5. The kit of any of claims 1-4, wherein an antibody-based moiety: biomarker protein complex is formed when the solid support is contacted with a sample.

6. The kit of any of claims 1-4, wherein at least one of said antibody-based moieties is immobilized on at least one solid support.

7. The kit of claim 6, wherein each of said antibody-based moiety is immobilized on the same solid support.

8. The kit of any of claims 1-4, wherein the labeled antibody-based moieties are detectably labeled and detectable label is selected from a group consisting of enzyme, fluorescent, biotin, gold, latex, hapten and radioisotope labeling.

9. A method for facilitating the diagnosis of a subject for chronic prostatitis/chronic pain pelvic syndrome (CP/CPPS) comprising:
a. measuring the level of at least one biomarker protein in a post-prostatic massage urine test sample obtained from the patient; and
b. comparing the level of the biomarker measured in the test sample with the level of the same biomarker protein present in a control/reference sample;
wherein a higher level of the measured biomarker protein in the test sample as compared to the level of the same biomarker protein in the control/reference sample is indicative of CP/CPPS, and wherein the biomarker protein is selected form the group consisting of uromodulin (THP), aminopeptidase N (AMPN), dipeptidylpeptidase IV (CD26), neprilysin (NEP), zinc-α-2-glycoprotein (ZA2G) and alkaline phosphatase (ALP); wherein higher levels of at least one biomarker protein in the test sample as compared to the level of biomarker proteins in the control/reference sample is indicative of CP/CPPS

10. The method of claim 9, wherein the presence of the biomarker protein is detected using an antibody-based moiety which specifically binds to the biomarker protein.

11. The method of claim 9, wherein the levels of at least 2, 3, 4, 5, or 6 biomarker proteins are measured and higher levels of at least 2, 3, 4, 5, or 6 biomarker proteins in the test sample as compared to the levels of biomarker proteins in the control/reference sample is indicative of CP/CPPS

12. The method of claim 10, wherein the level of biomarker protein is measured by a method comprising the steps of:
a. contacting the test sample, or preparation thereof, with an antibody-based binding moiety which specifically binds the biomarker protein to form an antibody-based binding moiety:biomarker protein complex; and
b. detecting the presence of the complex, thereby measuring the level of biomarker protein present.

13. The method according to claim 12, wherein the antibody-based binding moiety is labeled with a detectable label.

14. The method according to claim 13, wherein the label is selected from the group consisting of a radioactive label, a hapten label, a fluorescent label, and an enzymatic label.
